SEQUENCE LISTING (4 sequences)

SEQ ID NO: 1 - protein, 242 aa, Salmonella typhi
MKKLIFLTLSIVSFNNYAVDFVYRVDSTPPDVIFRDGFSLLGYNRNFQQFISGRSCSGGSSDSRYIATTSSVNQTYAIARAYYSRSTFKGNLYRYQIRADNNFYSLLPSITYLETQGGHFNAYEKTMMRLQREYVSTLSILPENIQKAVALVYDSATGLVKDGVSTMNASYLGLSTTSNPGVIPFLPEPQTYTQQRIDAFGPLISSCFSIGSVCHSHRGQRADVYNMSFYDARPVIELILSK

SEQ ID NO: 2 - protein, 137 aa, Salmonella typhi
MYMSKYVPVYTLLILIYSFNASAEWTGDNTNAYYSDEVISELHVGQIDTSPYFCIKTVKANGSGTPVVACAVSKQSIWAPSFKELLDQARYFYSTGQSVRIHVQKNIWTYPLFVNTFSANALVGLSSCSATQCFGPK

SEQ ID NO: 3 - protein, 141 aa, Salmonella typhi
MKKKLKVLTLALASISSVCYAAMADYDTYVSNVQINNLSYGVYTSGGKETQFFCIGLKHGSEAISINAMCKVDVYGNHKQGFDNMLNTAKYYYTTGGDVRIYYKENVWRDPDFKSAFSSRELIAITTCSSSSYCMGPTVTN

SEQ ID NO: 4 - protein, 269 aa, Salmonella typhi
MKKPVFFLLTMIICSYISFACANISDYKVMTWNLQGSSASTESKWNVNVRQLLSGTAGVDILMVQEAGAVPTSAVPTGRHIQPFGVGIPIDEYTWNLGTTSRQDIRYIYHSAIDVGARRVNLAIVSRQRADNVYVLRPTTVASRPVIGIGLGNDVFLTAHALASGGPDAAAIVRVTINFFRQPQMRHLSWFLAGDFNRSPDRLENDLMTEHLERVVAVLAPTEPTQIGGGILDYGVIVDRAPYSQRVEALRNPQLASDHYPVAFLARSC

(19) United States
(12) Patent Application Publication
(10) Pub. No.: US 2023/0263876 A1
(43) Pub. Date: Aug. 24, 2023

(54) COMPOSITIONS AND METHODS RELATING TO TYPHOID TOXIN SUBUNIT PLTC

(71) Applicant: YALE UNIVERSITY, New Haven, CT (US)

(72) Inventor: Jorge E. Galan, New Haven, CT (US)

(21) Appl. No.: 18/005,444

(22) PCT Filed: Jul. 15, 2021
(86) PCT No.: PCT/US21/41788

§ 371 Date: Jan. 13, 2023

Related U.S. Application Data
(60) Provisional application No. 63/052,684, filed on Jul. 16, 2020.

(51) Int. Cl. A61K 39/112; C07K 16/12; G01N 33/569

(52) U.S. Cl. CPC A61K 39/0275; C07K 16/1235; G01N 33/56916; A61K 2039/522

(57) ABSTRACT

The invention provides compositions and methods for preventing, treating and diagnosing infection by Salmonella enterica serovar typhi (S. typhi) and/or S. paratyphi, i.e., typhoid fever.

### CROSS REFERENCE TO RELATED APPLICATIONS

This application claims priority to U.S. Provisional Application No. 63/052,684, filed Jul. 16, 2020 which is hereby incorporated by reference herein in its entirety.

### BACKGROUND OF THE INVENTION

Salmonella enterica serovar typhi (S. typhi) and paratyphi (S. paratyphi), the causes of typhoid fever, results in more than 200,000 annual deaths (Parry et al., N. Engl. J. Med. 347:1770-1782; Crump et al., 2008, Antimicrob. Agents Chemother. 52:1278-1284; Raffatellu et al., 2008, J. Infect. Dev. Ctries. 2:260-266; Butler, 2011, Clin. Microbiol. Infect. 17:959-963; Crump and Mintz, 2010, Clin. Infect. Dis. 50:241-246). Unlike other Salmonella serovars, which typically cause self-limiting gastroenteritis, S. typhi and S. paratyphi cause a systemic, life-threatening disease (Parry et al., N. Engl. J. Med. 347:1770-1782). The genomes of S. typhi and S. paratyphi contain a dearth of unique virulence factors that are not found in non-typhoidal serovars, and the molecular bases for its unique virulence properties and clinical presentation are unknown (Sabbagh et al., 2010, FEMS Microbiol. Lett. 305:1-13; Parkhill et al., 2001, Nature 413:848-852).

One of the few S. typhi- and S. paratyphi-specific factors that has been shown to directly impact its interaction with host cells is an AB-type toxin dubbed typhoid toxin (Haghjoo and Galan, 2004, Proc. Natl. Acad. Sci. USA 101:4614-4619; Spano et al., 2008, Cell Host Microbe 3:30-38; Spano and Galan, 2008, Curr. Opin. Microbiol. 11:15-20). AB family toxins consists of enzymatically active A subunits that interfere with host functions, and B subunits that deliver the toxins to their target cells through receptor-mediated endocytosis (Beddoe et al., 2010, Trends Biochem. Sci. 35:411-418; Merritt and Hol, 1995, Curr. Opin. Struct. Biol. 5:165-171). Unlike typical AB toxins, typhoid toxin is composed of two A subunits, PltA and CdtB, which are homologs of the A subunits of the pertussis and cytolethal distending toxins, respectively (Spano et al., 2008, Cell Host Microbe 3:30-38).

There are currently no effective vaccines to protect against typhoid fever, and in particular to protect young children, the most susceptible population, against typhoid fever. Moreover, there are currently no effective and specific diagnostic tools for typhoid fever, and multiple antibiotic resistant S. typhi is rapidly emerging, with the prospects of typhoid fever being untreatable by antibiotics becoming a real threat (Butler, 2011, Clin. Microbiol. Infect. 17:959-963).

There are currently no WHO prequalified vaccines considered suitable for widespread use to protect against typhoid fever, and in particular to protect young children, the most susceptible population. Furthermore, the major strategy of recent vaccine efforts has been directed towards the Vi antigen surface polysaccharide, which is exclusively encoded by S. typhi. Consequently, there are no vaccines currently available to protect against S. paratyphi A, which does not encode Vi antigen and is estimated to be responsible for as much as ˜20-50% of all enteric fever cases.

Thus, there is a need in the art for compositions and methods for preventing, treating and diagnosing typhoid fever. The present invention addresses this unmet need.

### SUMMARY OF THE INVENTION

In one aspect, the present invention provides a composition for inducing or enhancing an immune response comprising PltC or a PltC mutant. In one embodiment, the composition further comprises at least one polypeptide selected from the group consisting of: a.) PltA, or a PltA mutant; b.) PltB, or a PltB mutant; and c.) CdtB or a CdtB mutant.

In one embodiment, the PltA mutant is PltA E133A, relative to SEQ ID NO: 1. In one embodiment, the PltB mutant is PltB S35A, relative to SEQ ID NO: 2. In one embodiment, the CdtB mutant is CdtB H160Q, relative to SEQ ID NO: 4; CdtB R119X, relative to SEQ ID NO: 4; CdtB H259X, relative to SEQ ID NO: 4; CdtB ΔCys269, relative to SEQ ID NO: 4; or CdtB C269X, relative to SEQ ID NO: 4. In one embodiment, the PltA mutant comprises any mutation in PltA that disrupts its enzymatic activity. In one embodiment, the CdtB mutant comprises any mutation in CdtB that disrupts its enzymatic activity.

In one embodiment, the composition comprises PltA, or a PltA mutant; CdtB, or a CdtB mutant; and PltC or a PltC mutant.

In one embodiment, the composition further comprises an antigen, wherein the composition enhances the immune response against the antigen. In one embodiment, the antigen is selected from the group consisting of a bacterial antigen, viral antigen, parasitic antigen, cancer antigen, tumor-associated antigen, and tumor-specific antigen. In one embodiment, the antigen is a S. typhi antigen or a S. paratyphi antigen. In one embodiment, the antigen is a bacterial polysaccharide antigen. In one embodiment, the antigen is Vi antigen.

In one embodiment, the composition comprises a vaccine.

In one embodiment, the composition comprises a bacterium.

In one aspect, the present invention provides a method of inducing an immune response in a subject, the method comprising administering a composition comprising PltC or a PltC mutant to the subject.

In one embodiment, the subject is currently infected with S. typhi or S. paratyphi and the composition induces an immune response against S. typhi or S. paratyphi. In one embodiment, the subject is not currently infected with S. typhi or S. paratyphi and the composition induces an immune response against S. typhi or S. paratyphi.

In one aspect, the present invention provides a method of treating or preventing S. typhi infection in a subject, comprising administering a composition comprising PltC or a PltC mutant to the subject. In one embodiment, the method further comprises administering an antibiotic to the subject.

In one aspect, the present invention provide a method for treating or preventing a disease or disorder associated with an antigen in a subject, comprising administering a composition comprising PltC or a PltC mutant, and an antigen, to the subject. In one embodiment, the disease or disorder is at least one selected from the group consisting of cancer, a bacterial infection, a viral infection, and a parasitic infection.

In one aspect, the present invention provides an inhibitor composition useful for treating or preventing S. typhi or S. paratyphi infection, wherein the inhibitor composition inhibits PltC. In one embodiment, the inhibitor composition comprises an antibody that specifically binds to PltC.

In one aspect, the present invention provides a method of treating a subject infected with S. typhi, the method comprising administering to the subject an inhibitor composition that inhibits PltC. In one embodiment, the method further comprises administering an antibiotic to the subject.

In one aspect, the present invention provides a method of diagnosing an S. typhi or S. paratyphi infection in a subject in need thereof, the method comprising: a.) determining the level of PltC in a biological sample of the subject, b.) comparing the level of PltC with level in a comparator control, and c.) diagnosing the subject with an infection by S. typhi or S. paratyphi when the level of PltC is significantly different when compared with the level in the comparator control. In one embodiment, the level of PltC in the biological sample is determined by measuring the level of PltC mRNA in the biological sample. In one embodiment, the level of PltC in the biological sample is determined by measuring the level of PltC polypeptide in the biological sample. In one embodiment, the comparator control is at least one selected from the group consisting of: a positive control, a negative control, a historical control, a historical norm, or the level of a reference molecule in the biological sample. In one embodiment, the method further comprises the step of administering a therapy to the subject to treat the infection.

In one aspect, the present invention provides a method of diagnosing an S. typhi or S. paratyphi infection in a subject in need thereof, the method comprising: a.) determining the level of antibody that specifically binds to PltC in a biological sample of the subject, b.) comparing the level of antibody that specifically binds to PltC with level in a comparator control, and c.) diagnosing the subject with an infection by S. typhi or S. paratyphi when the level of the antibody that specifically binds to PltC is significantly different when compared with the level in the comparator control. In one embodiment, the comparator control is at least one selected from the group consisting of: a positive control, a negative control, a historical control, a historical norm, or the level of a reference molecule in the biological sample. In one embodiment, the method further comprises the step of administering a therapy to the subject to treat the infection.

In one aspect, the present invention provides composition comprising a toxin-deficient S. typhi or S paratyphi bacterium, wherein the bacterium lacks PltC. In one embodiment, the composition is a vaccine and induces an adaptive immune response.

In one aspect, the present invention provides a method of immunizing a subject against S. typhi or S. paratyphi, the method comprising administering a composition comprising a toxin-deficient S. typhi or S paratyphi bacterium, wherein the bacterium lacks PltC, to the subject.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1F, depicts the results of exemplary experiments demonstrating that S. typhi produces two distinct typhoid toxins with common active but different delivery subunits. FIG. 1A: Illustration of the S. typhi typhoid toxin genomic locus, as well as a distant locus that encodes pltC (sty1364), an orphan pertussis-like toxin delivery subunit that exhibits homology to pltB. FIG. 1B and FIG. 1C: Expression of pltC, pltB and cdtB over time under conditions that stimulate typhoid toxin gene expression. The β-galactosidase activity in the pltC:lacZ, pltB:lacZ and cdtB:lacZ S. typhi reporter strains was measured at the indicated time points following infection of Henle-407 cells (FIG. 1B) or growth in TTIM medium (FIG. 1C). Values indicate the mean±S.D. for three independent samples. FIG. 1D: Interaction of PltC with PltA/CdtB in S. typhi grown in TTIM. Cell lysates from S. typhi strains encoding cdtB-3×FLAG, p/tC-3×FLAG, cdtB-3×FLAG (in ΔpltA background), or malE-3×FLAG were immunoprecipitated with an anti-FLAG antibody and interacting proteins were identified using LC/MS/MS. For each sample the number of peptides for the five most abundant proteins recovered and for all typhoid toxin subunits (color-coded according to FIG. 1A) are shown. *Detection of PltB with the LC-MS/MS protocol even for purified typhoid toxin preparations is inefficient. FIG. 1E: S. typhi produces both PltB- and PltC-typhoid toxins within infected human cells. Henle-407 cells were infected with S. typhi wild type or the indicated mutant strains encoding 3×FLAG epitope-tagged CdtB or PltC (as indicated) and 24 hrs post-infection the interaction of the indicated toxin components were probed by co-immunoprecipitation and western blot analysis. FIG. 1F: PltB forms a complex with CdtB, but not with PltC. The interaction of the indicated toxin components in cell lysates of the indicated strains encoding 3×FLAG epitope tagged CdtB or PltC were probed by anti-FLAG co-immunoprecipitation and western blot analysis. Whole cell lysates (Pre IP) and immunoprecipitated samples (post IP) were probed using an anti-FLAG antibody as a control (top blot) and an anti-PltB antibody (bottom blot) to identify PltB interactions with CdtB or PltC in the indicated strains. Ig. l. c.: Immunoglobulin light chain detected by the secondary antibody.

FIG. 2, comprising FIG. 2A through FIG. 2H, depicts the results of experiments demonstrating that the PltB- and PltC-typhoid toxins exhibit different biological properties. FIG. 2A: Gel filtration chromatography and SDS-PAGE/Coomassie blue (inset) analyses of purified PltC-typhoid toxin. Lanes on gel represent individual chromatographic fractions (red box) containing purified toxin. FIG. 2B: PltC-typhoid toxin elicits G2/M cell cycle arrest in human epithelial cells. Purified PltB- or PltC-typhoid toxins were added to the culture medium of Henle-407 cells at the indicated concentrations and 48 h after, cells were fixed and analyzed by flow cytometry to evaluate toxicity as indicated elsewhere herein. The data shown are the mean normalized toxicity±S.D. for three independent experiments. FIG. 2C: PltC-typhoid toxin does not induce G2/M arrest in S. typhi-infected cells. Henle-407 cells were infected with the indicated strains at a multiplicity of infection (MOI) of 10 or 30, as indicated, and 48 h post-infection cells were collected and the percentage of cells in G2/M phase was determined as described for FIG. 2B. Mean values±S.D. are shown for three independent experiments assayed in duplicate (6 total samples). Asterisks denote statistically significant levels G2/M cell cycle arrest compared to mock infected cells (red dotted line) as determined by unpaired two-tailed t-tests. FIG. 2D and FIG. 2E: PltC-typhoid toxin is not packaged into vesicle transport carriers. Henle-407 cells were infected with the indicated S. typhi strains encoding 3×-FLAG epitope-tagged CdtB and 48 hrs post-infection the cells were fixed and stained with DAPI (blue), α-FLAG (green), and α-S. typhi LPS (red) antibodies. Typhoid toxin-containing export vesicles, which appear as green puncta (FIG. 2D), were quantified by image analysis (FIG. 2E) as indicated elsewhere herein. Values are from >25 images (˜100 infected cells) taken in two independent experiments and represent the mean relative ratios±S.E.M. Asterisks denote the statistical significance of the indicated pairwise comparisons determined using unpaired two-tailed t-tests. FIG. 2F-FIG. 2H: The PltB- and PltC-typhoid toxins elicit different effects when administered to mice. Highly purified preparations of PltB-(2 μg) or PltC-typhoid toxins (10 μg) were administered to C57BL/6 mice. For one group of mice, their survival (FIG. 2F) and body weight (mean±S.D.) (FIG. 2G) was recorded at the indicated times. The remaining mice were killed at four days post-toxin administration and a blood sample was collected and analyzed to quantify the indicated cell types (mean±S.D.) (FIG. 2H). WBCs, white blood cells. The Mantel-Cox test was used for statistical analysis of mouse survival and Brown-Forsythe and Welch ANOVA coupled with Dunnett's T3 multiple comparisons tests were used to statistically compare the indicated samples for the blood analysis. For all panels, ****p<0.0001, ***p<0.001, **p<0.01, *p<0.05, n.s.s. not statistically significant.

FIG. 3, comprising FIG. 3A through FIG. 3F, depicts the results of exemplary experiments demonstrating that distinct regulatory mechanisms and metabolic cues control the expression of PltB and PltC. FIG. 3A: Schematic representation of the FAST-INseq genetic screen used to identify S. typhi genes that influence pltC expression in infected host cells. A large library of random transposon mutants was generated in the S. typhi pltC:gfp strain and used to infect Henle-407 cells. Sixteen hours post-infection the bacteria were isolated and sorted by FACS into pools exhibiting high and low GFP fluorescence. INseq was then used to identify mutants that stimulate (green dot in plot) or reduce (red dot) pltC expression during infection. FIG. 3B: Overview of the results of the FAST-INseq screen. Plot shows the normalized numbers of sequencing reads of transposon insertions within each S. typhi gene in the high fluorescence vs. low fluorescence pools. FIG. 3C and FIG. 3D: Expression levels of pltB:lacZ and pltC:lacZ reporters in infected human cells for wild-type S. typhi (WT) and the indicated deletion mutant strains. Henle-407 cells were infected with the indicated strains for 24 h, after which the β-galactosidase activity from bacterial lysates was measured and normalized to the numbers of CFU recovered. Values indicate mean values±S.D. for six independent determinations taken over two separate experiments. Asterisks denote statistically significant differences relative to the corresponding wild-type sample determined using unpaired two-tailed t-tests. ****p<0.0001, *p<0.05, n.s.s. not statistically significant. FIG. 3E: Flow cytometry analysis of pltB:gfp and pltC:gfp expression of the indicated S. typhi strains 24 h post-infection. Histograms show the GFP fluorescence intensities of individual bacteria for the indicated strains. Gates were established to show the percentage of bacteria exhibiting high, low and intermediate (int) fluorescence. The percentage of bacteria with fluorescence intensities within these gates is shown (bottom). Gating strategy provided in FIG. 9B. FIG. 3F: Overview of the identified factors that differentially affect the expression of pltB and pltC and thus are likely to be important for controlling relative abundance of the two typhoid toxins produced by S. typhi upon encountering different environments during infection.

FIG. 4 depicts the congruent phylogenetic distributions of the typhoid toxin islet and a second pertussis toxin-like B subunit homolog in the Salmonella genus. Depiction of different genomic arrangements of the typhoid toxin islet and of loci encoding a second putative B subunit that were identified by searching the NCBI genome database. All genomic locations are shown relative to the S. typhi reference genome (black arrows). Large genomic islands absent from the typhi genome are shown in yellow, pseudogenes are shown as grey boxes and ADP-ribosyltransferase (ADP-RT) pseudogenes have a red outline. The depicted genomic arrangements appear, based on the available sequence data, to be conserved across the cited species (Sp.), subspecies (Ssp.), clade, serovar (Sv.) or strains, as indicated.

FIG. 5 depicts the amino acid sequence comparison between S. typhi PltB and PltC (Sty1364). A pairwise global alignment was done using the EMBL-EBI Matcher program.

FIG. 6 depicts the results of example experiments demonstrating that deletion of pltC results in increased production of PltB-typhoid toxin. Two CdtB-3×FLAG encoding S. typhi strains, wild type and ΔpltC, were grown in TTIM to induce typhoid toxin expression. Cell lysates were immunoprecipitated with an anti-FLAG antibody and the abundance of PltB and CdtB in the eluates was measured by western blot using anti-PltB and anti-FLAG antibodies, respectively. The abundance of PltB in the wild-type and ΔpltC samples was normalized to the CdtB levels and compared using a one-sample t-test to determine if PltB abundance in the ΔpltC sample deviated from a theoretical value of 100% of the wild-type value. Mean values+/−S.D. are shown for 3 independent experiments. * p<0.05.

FIG. 7 depicts the results of example experiments demonstrating that both typhoid toxins are secreted using the same TtsA-dependent secretion mechanism. The mechanism of typhoid toxin secretion was recently described, in which the activity of the specialized muramidase TtsA enables the toxin to access the trans side of the peptidoglycan layer, after which it can be released following exposure to outer membrane perturbing agents such as bile salts (Geiger et al., 2018, Nat. Microbiol., 3: 1243-1254). To determine whether this mechanism is utilized by both PltC-typhoid toxin and PltB-typhoid toxin, an in vitro secretion assay was performed (Geiger et al., 2018, Nat. Microbiol., 3: 1243-1254). The indicated strains, all of which encode a 3×FLAG-epitope tagged CdtB, were grown for 24 hours in TTIM medium to induce typhoid toxin (and ttsA) expression, after which the bacteria were pelleted and washed thoroughly. The material was evenly divided between two samples, one of which was incubated in PBS containing 0.075% bile salts (B.S.) and the other in PBS only. Samples were then pelleted and the levels of CdtB-3×FLAG in the pellet fraction and in filtered culture supernatants (Sup.) were analyzed by western blot.

FIG. 8 depicts the results of example experiments demonstrating that expression of pltC by intracellular S. typhi, unlike the other typhoid toxin genes, does not require the PhoP/PhoQ two-component system. Henle-407 cells were infected using wild type and ΔphoP/phoQ mutant S. typhi strains that encode 3×FLAG epitope-tagged versions of both cdtB and pltC at their native genomic loci, as well as an untagged control strain. Whole cell lysates from the inoculum used for the infections as well as the bacteria isolated from infected cells 24 hours post-infection were analyzed by western blot. Sample loading was normalized to CFU recovery and lysate from 2.5×10⁷ bacteria was loaded in each lane.

FIG. 9, comprising FIG. 9A and FIG. 9B, depicts the results of example experiments showing single bacterium-level expression of the typhoid toxin B subunits by S. typhi within infected cells in strains lacking key two-component regulators of intracellular gene expression. FIG. 9A: The SsrA/SsrB, PhoP/PhoQ and EnvZ/OmpR two-component regulatory systems are all central regulators of intracellular gene expression and all were identified as leading to reduced pltC expression in the FAST-INseq screen (Table 3). To probe the role of these regulators in the expression of pltB and pltC, a flow cytometry analysis of pltB:gfp and pltC:gfp expression was conducted of the indicated S. typhi strains 24 hours post-infection. Histograms show the GFP fluorescence intensities of individual bacteria. For both wildtype strains, samples show high fluorescence (most bacteria) and low fluorescence (rare bacteria) populations; gates were established to show the percentage of bacteria in these populations for each strain, as well as those with an intermediate level of fluorescence (“int”) and those with a level greater than 99.9% of the wild-type population (>high). The proportions of bacteria with fluorescence intensities within these gates is shown (bottom). The heterogeneous expression of pltC in the ΔphoP/phoQ and ΔenvZ/ompR strains is likely due to their influence over ssrA/ssrB expression (Fass et al., 2009, Curr Opin Microbol., 12: 199-204; Lee et al., 2000, J. Bacteriol, 182: 771-781). FIG. 9B: Gating strategy used for flow cytometry to analyze S. typhi typhoid toxin gene expression at the single bacterium level during infection (FIG. 9A and FIG. 3E). Strains carried a low copy plasmid that constitutively expresses mCherry. To identify particles that represent S. typhi (as opposed to cell debris remaining from host cell lysis), only particles with an mCherry fluorescence level above background were analyzed. An example of this gating (pltC:GFP wild-type strain) is shown.

FIG. 10, comprising FIG. 10A and FIG. 10B, depicts examples of AB5-type toxins and orphan homologous delivery subunits encoded within the same genome outside the Salmonella genus. Using BLAST homology searches using AB5-type toxin subunits, genomes encoding an AB5-type toxin as well as a homologous orphan delivery subunit were identified. FIG. 10A: In addition to the five-gene pertussis toxin locus (Gross et al., 1989, Mol Microbiol, 3: 119-124), it was found that B. pertussis encodes two putative AB5 toxin delivery subunits at a distant genomic locus that does not also encode a putative toxin active subunit, bp1251 and bp1252. Bp1251 is >30% identical to both PtxB and PtxC over >200 amino acids. Bp1252 was identified as a statistically significant hit (e value <0.02) to PtxB, PtxC and PtxE using the HHPred protein homology detection tool (https://toolkit.tuebingen.mpg.de/#/tools/hhpred). FIG. 10B: The putative AB5-type toxin Yersinia toxin (Axler-Diperte et al., 2006, J Bacteriol, 188: 8033-8043) is encoded by ytxA and ytxB in Y. enterocolitica. At a distant genomic location, Y. enterocolitica also encodes an orphan pertussis toxin-like delivery subunit, ye1123A. YtxA and Ye1123A exhibit significant sequence similarity to the pertussis-like toxin ArtAB; YtxA is >60% identical to ArtA and Ye1123A is ˜30% identical to ArtB.

### DETAILED DESCRIPTION

The present invention provides compositions and methods for inducing or enhancing an immune response. For example, in certain embodiments, the invention relates to inducing or enhancing cell-mediated and/or humoral immunity directed against a desired antigen.

In certain embodiments, the compositions and methods are used to prevent, treat and diagnose infection by Salmonella enterica serovar typhi (S. typhi) and/or S. paratyphi, i.e., typhoid fever. In one embodiment, the composition of the invention is a vaccine that induces the cell-mediated and/or humoral immunity directed against at least one S. typhi and/or S. paratyphi protein (e.g., PltA, PltB, PltC, CdtB, etc). In one embodiment, the composition comprises PltA, or a mutant thereof. In one embodiment, the composition comprises PltB, or a mutant thereof. In one embodiment, the composition comprises PltC, or a mutant thereof. In one embodiment, the composition comprises CdtB, or a mutant thereof.

In another embodiment, the composition of the invention comprises a nucleic acid sequence encoding PltA, or a mutant thereof. In another embodiment, the composition of the invention comprises a nucleic acid sequence encoding PltB, or a mutant thereof. In another embodiment, the composition of the invention comprises a nucleic acid sequence encoding PltC, or a mutant thereof. In one embodiment, the composition of the invention comprises a nucleic acid sequence encoding CdtB, or a mutant thereof.

In one embodiment, the composition comprises a bacterium comprising one or more of PltA, PltB, PltC, CdtB, or a mutant thereof. In one embodiment, the composition comprises a bacterium comprising one or more nucleic acids encoding one or more of PltA, PltB, PltC, CdtB, or a mutant thereof. In one embodiment, the composition comprises a bacterium in which one or more of PltA, Pltb, PltC, CtdB, or a mutant thereof, are absent (i.e., toxin deficient). For example, in one embodiment, the composition comprises a S. typhi or a S. paratyphi bacterium.

In one embodiment, at least one of PltA, PltB, PltC, CdtB, or a mutant thereof of the composition, or expressed by the composition, serves as an antigen to induce immunity directed against at least one of PltA, PltB, PltC, or CdtB. In another embodiment, at least one of PltA, PltB, PltC, CdtB, or a mutant thereof of the composition, or expressed by the composition, serves as an adjuvant to enhance immunity directed against an antigen. In various embodiments, the antigen is from a virus, a bacteria, a parasite, or a cancer cell. In some embodiments, the antigen is a S. typhi or S. paratyphi antigen. In other embodiments, the antigen is not an S. typhi or S. paratyphi antigen.

In one embodiment, the composition comprises an antibody that specifically binds to PltA, or a mutant thereof. In one embodiment, the composition comprises an antibody that specifically binds to PltB, or a mutant thereof. In one embodiment, the composition comprises an antibody that specifically binds to PltC, or a mutant thereof. In one embodiment, the composition comprises an antibody that specifically binds to CdtB, or a mutant thereof. In one embodiment, the composition comprises an antibody that specifically binds to a toxin comprising one or more of PltA, PltB, PltC, or CdtB.

The invention provides methods of inducing an immune response for preventing or treating infection by S. typhi or S. paratyphi. In one embodiment, the methods comprise administering at least one of PltA, PltB, PltC, CdtB, or a mutant thereof, to a subject. In another embodiment, the methods comprise administering a nucleic acid encoding at least one of PltA, PltB, PltC, CdtB, or a mutant thereof, to a subject.

The invention provides a method of inducing an immune response for preventing or treating an infection, disease, or disorder associated with an antigen. In one embodiment, the methods comprise administering an antigen and at least one of PltA, PltB, PltC, CdtB, or a mutant thereof, to a subject.

In another embodiment, the methods comprise administering a nucleic acid encoding and expressing at least one of PltA, PltB, Pltc, CdtB, or a mutant thereof, to a bacterium. In some embodiments, the bacterium already comprises a nucleic acid that encodes, but does not express, PltA, PltB, PltC, CdtB, or a mutant thereof. In other embodiments, the bacterium already comprises a nucleic acid that encodes, and does express, PltA, PltB, PltC, CdtB, or a mutant thereof.

The invention also provides methods of treating infection by S. typhi or S. paratyphi in a subject in need thereof. In one embodiment, the method comprises administering to the subject at least one antibody that specifically binds to PltA, or a mutant thereof. In one embodiment, the method comprises administering to the subject at least one antibody that specifically binds to PltB, or a mutant thereof. In one embodiment, the method comprises administering to the subject at least one antibody that specifically binds to PltC, or a mutant thereof. In one embodiment, the method comprises administering to the subject at least one antibody that specifically binds to CdtB, or a mutant thereof. In one embodiment, the method comprises administering to the subject at least two antibodies that specifically bind to at least two of PltA, PltB, PltC, and CdtB, or a mutant thereof. In one embodiment, the method comprises administering to the subject at least one antibody that specifically binds to a toxin comprising one or more of PltA, PltB, PltC, or CdtB.

The invention also includes inhibitor compositions and methods for inhibiting with the interaction between the S. typhi or S. paratyphi toxin and the toxin's receptor.

The invention also provides methods of diagnosing infection by S. typhi or S. paratyphi in a subject by detecting the presence of, or measuring the level of, in the subject, at least one of PltA, PltB, PltC, or CdtB, or antibodies that specifically bind to at least one of PltA, PltB, PltC, CdtB, or a mutant thereof. In one embodiment, the method comprises detecting the presence of, or measuring the level of, in the subject, a toxin comprising one or more of PltA, PltB, PltC, or CdtB, or antibodies that specifically bind to a toxin comprising one or more of PltA, PltB, PltC, or CdtB or a mutant thereof.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample.

As used herein, the term “autologous” is meant to refer to any material derived from an individual to which it is later to be re-introduced into the same individual.

The term “adjuvant” as used herein is defined as any molecule to enhance an antigen-specific adaptive immune response.

The term “agent” includes any substance, metabolite, molecule, element, compound, or a combination thereof. It includes, but is not limited to, e.g., protein, oligopeptide, small organic molecule, glycan, polysaccharide, polynucleotide, and the like. It can be a natural product, a synthetic compound, a chemical compound, or a combination of two or more substances. Unless otherwise specified, the terms “agent,” “substance,” and “compound” can be used interchangeably. Further, a “test agent” or “candidate agent” is generally a subject agent for use in an assay of the invention.

The term “binding” refers to a direct association between at least two molecules, due to, for example, covalent, electrostatic, hydrophobic, ionic and/or hydrogen-bond interactions.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

A “chimeric antibody” refers to a type of engineered antibody which contains a naturally-occurring variable region (light chain and heavy chains) derived from a donor antibody in association with light and heavy chain constant regions derived from an acceptor antibody.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components.

As used herein, by “combination therapy” is meant that a first agent is administered in conjunction with another agent. “In conjunction with” refers to administration of one treatment modality in addition to another treatment modality. As such, “in conjunction with” refers to administration of one treatment modality before, during, or after delivery of the other treatment modality to the individual. Such combinations are considered to be part of a single treatment regimen or regime.

As used herein, the term “concurrent administration” means that the administration of the first therapy and that of a second therapy in a combination therapy overlap temporally with each other.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

An “effective amount” as used herein, means an amount which provides a therapeutic or prophylactic benefit.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of VH (variable heavy chain immunoglobulin) genes from an animal.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared multiplied by 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

The term “immunoglobulin” or “Ig,” as used herein, is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “immune response” includes T-cell mediated and/or B-cell mediated immune responses. Exemplary immune responses include T cell responses, e.g., cytokine production and cellular cytotoxicity, and B cell responses, e.g., antibody production. In addition, the term immune response includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

As used herein, an “inhibitory-effective amount” is an amount that results in a detectable (e.g., measurable) amount of inhibition of an activity. In some instance, the activity is its ability to bind with another component.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “mutation,” as used herein, refers to a change in nucleic acid or polypeptide sequence relative to a reference sequence (which, in certain instances, is a naturally-occurring normal or “wild-type” sequence), and includes translocations, deletions, insertions, and substitutions/point mutations. A “mutant” as used herein, refers to either a nucleic acid or protein comprising a mutation.

“Parenteral” administration of an immunogenic composition includes, e.g., subcutaneous (s.c.), intravenous (i.v.), intramuscular (i.m.), or intradermal (i.d.) injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “X,” the presence of a molecule containing epitope X (or free, unlabeled A), in a reaction containing labeled “X” and the antibody, will reduce the amount of labeled X bound to the antibody.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “therapeutic” as used herein means a treatment and/or prophylaxis. A therapeutic effect is obtained by suppression, diminution, remission, or eradication of a disease state.

The term “therapeutically effective amount” refers to the amount of the subject compound that will elicit the biological or clinical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

A term “toxoid” as used herein, refers to a bacterial toxin, the toxicity of which has been inactivated or suppressed, such as by introduction of a mutation, a chemical treatment, or a heat treatment, while other properties of the toxin, such as immunogenicity, are maintained in the toxoid. In some literature, toxoids are referred to as anatoxins.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention relates to the administration of at least one of PltA, PltB, PltC, CdtB, or a mutant thereof, of S. typhi or and S. paratyphi to a subject to induce or enhance an immune response. Thus, the present invention provides a polypeptide or a combination of polypeptides, a polynucleotide or a combination of polynucleotides, which are useful in inducing or enhancing an immune response. In certain embodiments, at least one of PltA, PltB, PltC, CdtB, or a mutant thereof, is used to induce an immune response directed against PltA, PltB, PltC, CdtB, or any other S. typhi and/or S. paratyphi antigen (e.g., Vi antigen) for the treatment or prevention of infection by S. typhi or S. paratyphi. In certain embodiments, at least one PltA, PltB, PltC, CdtB, or a mutant thereof, is used to enhance an immune response directed against an antigen for the treatment or prevention of an infection, disease, or disorder associated with the antigen. Exemplary antigens include, but are not limited to, a viral antigen, a bacterial antigen, a parasitic antigen, a cancer antigen, a tumor-associated antigen, and a tumor-specific antigen.

The invention provides an immunological composition comprising a polypeptide or combination of polypeptides derived from at least one of PltA, PltB, PltC, CdtB, or a mutant thereof, useful in eliciting or enhancing an immune response. The compositions comprising one or more polypeptides of the invention not only are useful as a prophylactic therapeutic agent for immunoprotection, but are also useful as a therapeutic agent for treatment of an ongoing infection, disease, or disorder associated with an antigen.

In certain instances, one or more of PltA, PltB, PltC, CdtB, or a mutant thereof, serve as an antigen, to which the immune response is directed. In certain instances, one or more of PltA, PltB, PltC, CdtB, or a mutant thereof serve as an adjuvant to enhance an immune response directed against an antigen.

In one embodiment, the composition of the invention comprises a nucleic acid sequence encoding PltA, or a mutant thereof. In one embodiment, the PltA mutant is PltA E133X. In another embodiment, the PltA mutant is PltA E133A.

In one embodiment, the composition of the invention comprises a nucleic acid sequence encoding PltB, or a mutant thereof. In one embodiment, the PltB mutant is PltB S35X. In another embodiment, the PltB mutant is PltB S35A.

In one embodiment, the composition of the invention comprises a nucleic acid sequence encoding PltC, or a mutant thereof.

In one embodiment, the composition of the invention comprises a nucleic acid sequence encoding CdtB, or a mutant thereof. In one embodiment, the CdtB mutant is CdtB H160X. In another embodiment, the CdtB mutant is CdtB H160Q. In one embodiment, the CdtB mutant is CdtB R119X. In one embodiment, the CdtB mutant is CdtB H259X. In one embodiment, the CdtB mutant is CdtB ΔCys269. In another embodiment, the CdtB mutant is CdtB C269X.

In one embodiment, the mutant PltA comprises any mutation in PltA that disrupts its enzymatic activity. In one embodiment, the mutant PltB comprises any mutation in PltB that disrupts its enzymatic activity. In one embodiment, the mutant PltC comprises any mutation in PltC that disrupts its enzymatic activity. In one embodiment, the mutant CdtB comprises any mutation in CdtB that disrupts its enzymatic activity.

In one embodiment, the composition comprises a bacterium comprising at least one of PltA, PltB, PltC, CdtB, or a mutant thereof. In one embodiment, the composition comprises a bacterium comprising at least one nucleic acid encoding at least one of PltA, PltB, PltC, CdtB, or a mutant thereof. In one embodiment, the composition comprises a bacterium in which at least one of PltA, PltB, PltC, CtdB, or a mutant thereof, are absent (i.e. toxin deficient). For example, in one embodiment, the composition comprises a S. typhi or a S. paratyphi bacterium.

The skilled artisan will understand that the least one of PltA, PltB, PltC, CdtB, or a mutant thereof, useful in eliciting an immune response, can each be used alone or in any combination for eliciting an immune response. In one embodiment, the composition comprises a toxin comprising PltA, PltC and CdtB, or mutants thereof.

In one embodiment, the composition comprises at least one antigen. In one embodiment, the composition comprises at least one nucleic acid molecule encoding at least one antigen. For example, in one embodiment, the composition comprises an antigen and at least one of PltA, PltB, PltC, CdtB, or a mutant thereof. In one embodiment, the composition comprises at least one nucleic acid molecule encoding at least one antigen and at least one of PltA, PltB, PltC, CdtB, or a mutant thereof. Exemplary antigens include, but are not limited to, a viral antigen, a bacterial antigen, a parasitic antigen, a cancer antigen, a tumor-associated antigen, and a tumor-specific antigen.

The present invention also provides methods of preventing, inhibiting, and treating infection by S. typhi or S. paratyphi in a subject in need thereof. In one embodiment, the methods of the invention induce immunity against S. typhi or S. paratyphi in the subject, by generating an immune response in the subject directed to at least one polypeptide, such as PltA, PltB, PltC, and CdtB. In one embodiment, the methods of the invention induce production of PltA-specific antibodies in the subject. In one embodiment, the methods of the invention induce production of PltB-specific antibodies in the subject. In one embodiment, the methods of the invention induce production of PltC-specific antibodies in the subject. In one embodiment, the methods of the invention induce production of CdtB-specific antibodies in the subject. In one embodiment, the methods of the invention enhance the immune response directed against another S. typhi or S. paratyphi antigen, including but not limited to a bacterial polysaccharide antigen such as Vi antigen In one embodiment, the methods of the invention prevent S. typhi or S. paratyphi related pathology in a subject in need thereof. In one embodiment, the methods of the invention comprise administering to the subject a composition comprising at least a portion of at least one of PltA, PltB, PltC, CdtB, or a mutant thereof, to a subject. In another embodiment, the methods of the invention comprise administering to the subject a composition comprising a nucleic acid sequence encoding at least one of PltA, PltB, PltC, CdtB, or a mutant thereof, to a subject. In various embodiments, the composition can be comprise a single subunit of A₂B₅, a combination of subunits of A₂B₅, or the entire A₂B₅, wherein at least one of the subunits is a mutant subunit.

In one embodiment, the present invention provides methods of preventing, inhibiting, or treating an infection, disease, or disorder associated with an antigen. In one embodiment, the methods of the invention enhance an immune response against an antigen. Exemplary antigens include, but are not limited to, a viral antigen, a bacterial antigen, a parasitic antigen, a cancer antigen, a tumor-associated antigen, and a tumor-specific antigen.

In another embodiment, the methods of the invention comprise administering to the subject a bacterium or virus comprising a nucleic acid sequence encoding at least one of PltA, PltB, PltC, CdtB, or a mutant thereof. In another embodiment, the methods of the invention comprise administering to the subject a bacterium or virus expressing at least a portion of at least one of PltA, PltB, PltC, CdtB, or a mutant thereof. In another embodiment, the methods of the invention comprise administering to the subject a bacterium or virus comprising at least a portion of at least one of PltA, PltB, PltC, CdtB, or a mutant thereof.

The invention also includes inhibitor compositions and methods for inhibiting with the interaction between the S. typhi or S. paratyphi toxin and the toxin's receptor.

The invention also provides methods of diagnosing infection by S. typhi or S. paratyphi in a subject by detecting the presence of, or measuring the level of, in the subject, at least one of PltA, PltB, PltC, CdtB, or a mutant thereof, or antibodies that specifically bind to at least one of PltA, PltB, PltC, CdtB, or a mutant thereof.

### Compositions

The present invention provides compositions, including polypeptides, nucleotides, vectors, bacteria, and vaccines, that when administered to a subject, elicit or enhance an immune response. In certain instances the composition elicits an immune response directed against S. typhi or S. paratyphi, including an immune response directed against at least one of PltA, PltB, PltC, CdtB, or a mutant thereof. Further, when the compositions are administered to a subject, they elicit an immune response that serves to protect the inoculated subject against conditions associated with S. typhi or S. paratyphi infection. In certain embodiments, the compositions enhance an immune response directed against an antigen. For example, in certain aspects, PltA, PltB, PltC, CdtB, or a mutant thereof, serve as an adjuvant to enhance the immune response directed against a desired antigen. Exemplary antigens include, but are not limited to, a viral antigen, a bacterial antigen, a parasitic antigen, a cancer antigen, a tumor-associated antigen, and a tumor-specific antigen.

In one embodiment, the present invention provides compositions that are useful as immunomodulatory agents, for example, for stimulating immune responses and in preventing S. typhi or S. paratyphi related pathology. In various embodiments, the immunomodulatory agents comprise at least one of PltA, PltB, PltC, CdtB, or a mutant thereof. In one embodiment, the immunomodulatory agent comprises a complex (e.g. toxin) comprising PltA, PltC, and CdtB, or mutants thereof. In one embodiment, the immune response is not detrimental to the host and therefore the compositions of the invention are useful as a vaccine. In one embodiment, the immunomodulatory agents are administered in combination with an adjuvant. In another embodiment, the immunomodulatory agents are administered in the absence of an adjuvant.

In one embodiment, the compositions are useful as an adjuvant to enhance an immune response directed against a desired antigen for the prevention or treatment of an infection, disease, or disorder associated with the antigen. In one embodiment, the antigen is a bacterial polysaccharide. In one embodiment, the antigen is a S. typhi or S. paratyphi antigen, including but not limited to PltA, PltB, PltC, CdtB, and Vi antigen. In certain embodiments, the antigen is a viral antigen, bacterial antigen, parasitic antigen, cancer antigen, tumor-associated antigen, or tumor-specific antigen. However, the invention is not limited to any particular antigen. Rather, in various embodiments, the compositions may serve as an adjuvant to enhance an immune response directed against any desired antigen.

PltA, PltB, PltC, CdtB, or a mutant thereof can be, used as immunostimulatory agents to induce or enhance the production of specific antibodies. In certain aspects, the immunostimulatory agents protect against S. typhi or S. paratyphi induced pathology. Therefore, in one embodiment, the composition of the invention comprises a PltA polypeptide, or a mutant thereof. In one embodiment, the PltA mutant is PltA E133X. In one embodiment, the PltA mutant is PltA E133A. In one embodiment, the composition of the invention comprises a PltB polypeptide, or a mutant thereof. In one embodiment, the PltB mutant is PltB S35X. In one embodiment, the composition of the invention comprises a PltC polypeptide, or a mutant thereof. In one embodiment, the PltB mutant is PltB S35A. In one embodiment, the composition of the invention comprises a CdtB polypeptide, or a mutant thereof. In one embodiment, the CdtB mutant is CdtB H160X. In one embodiment, the CdtB mutant is CdtB H160Q. In one embodiment, the CdtB mutant is CdtB R119X. In one embodiment, the CdtB mutant is CdtB H259X. In one embodiment, the CdtB mutant is CdtB ΔCys269. In one embodiment, the CdtB mutant is CdtB C269X. The skilled artisan will understand that the least one of PltA, PltB, PltC, CdtB, or a mutant thereof, useful in eliciting an immune response, can each be used alone or in any combination for eliciting an immune response.

Exemplary PltA, PltB, PltC, and CdtB amino acid sequences are provided below. In certain embodiments, the PltA, PltB, PltC and CdtB mutants described herein are relative to the corresponding sequences below.

PltA; NP_456278
(SEQ ID NO: 1)
MKKLIFLTLSIVSFNNYAVDFVYRVDSTPPDVIFRDGFSLLGYNRNFQQ
FISGRSCSGGSSDSRYIATTSSVNQTYAIARAYYSRSTFKGNLYRYQIR
ADNNFYSLLPSITYLETQGGHFNAYEKTMMRLQREYVSTLSILPENIQK
AVALVYDSATGLVKDGVSTMNASYLGLSTTSNPGVIPFLPEPQTYTQQR
IDAFGPLISSCFSIGSVCHSHRGQRADVYNMSFYDARPVIELILSK
PltB; NP_456279.1
(SEQ ID NO: 2)
MYMSKYVPVYTLLILIYSFNASAEWTGDNTNAYYSDEVISELHVGQIDT
SPYFCIKTVKANGSGTPVVACAVSKQSIWAPSFKELLDQARYFYSTGQS
VRIHVQKNIWTYPLFVNTFSANALVGLSSCSATQCFGPK
PltC;
(SEQ ID NO: 3)
MKKKLKVLTLALASISSVCYAAMADYDTYVSNVQINNLSYGVYTSGGKE
TQFFCIGLKHGSEAISINAMCKVDVYGNHKQGFDNMLNTAKYYYTTGGD
VRIYYKENVWRDPDFKSAFSSRELIAITTCSSSSYCMGPTVTN
CdtB; NP_456275.1
(SEQ ID NO: 4)
MKKPVFFLLTMIICSYISFACANISDYKVMTWNLQGSSASTESKWNVNV
RQLLSGTAGVDILMVQEAGAVPTSAVPTGRHIQPFGVGIPIDEYTWNLG
TTSRQDIRYIYHSAIDVGARRVNLAIVSRQRADNVYVLRPTTVASRPVI
GIGLGNDVFLTAHALASGGPDAAAIVRVTINFFRQPQMRHLSWFLAGDF
NRSPDRLENDLMTEHLERVVAVLAPTEPTQIGGGILDYGVIVDRAPYSQ
RVEALRNPQLASDHYPVAFLARSC

In one embodiment, the composition of the invention comprises a PltA polypeptide comprising the amino acid sequence of SEQ ID NO: 1. In one embodiment, the composition of the invention comprises a PltB polypeptide comprising the amino acid sequence of SEQ ID NO: 2. In one embodiment, the composition of the invention comprises a PltC polypeptide comprising the amino acid sequence of SEQ ID NO: 3. In one embodiment, the composition of the invention comprises a CdtB polypeptide comprising the amino acid sequence of SEQ ID NO: 4.

The present invention also provides polynucleotides that encode the polypeptides described herein. Therefore, in one embodiment, the composition of the invention comprises a nucleic acid sequence encoding PltA, or a mutant thereof. In one embodiment, the PltA mutant is PltA E133X. In another embodiment, the PltA mutant is PltA E133A. In another embodiment, the composition of the invention comprises a nucleic acid sequence encoding PltB, or a mutant thereof. In one embodiment, the PltB mutant is PltB S35X. In another embodiment, the PltB mutant is PltB S35A. In another embodiment, the composition of the invention comprises a nucleic acid sequence encoding PltC, or a mutant thereof. In one embodiment, the composition of the invention comprises a nucleic acid sequence encoding CdtB, or a mutant thereof. In one embodiment, the CdtB mutant is CdtB H160X. In another embodiment, the CdtB mutant is CdtB H160Q. In one embodiment, the CdtB mutant is CdtB R119X. In another embodiment, the CdtB mutant is CdtB H259X. In one embodiment, the CdtB mutant is CdtB ΔCys269. In another embodiment, the CdtB mutant is CdtB C269X. The skilled artisan will understand that the least one of PltA, PltB, PltC, CdtB, or a mutant thereof, useful in eliciting an immune response, can each be used alone or in any combination for eliciting an immune response.

In one embodiment, the composition of the invention comprises a polynucleotide encoding a PltA polypeptide comprising the amino acid sequence of SEQ ID NO: 1. In one embodiment, the composition of the invention comprises a polynucleotide encoding a PltB polypeptide comprising the amino acid sequence of SEQ ID NO: 2. In one embodiment, the composition of the invention comprises a polynucleotide encoding a PltC polypeptide comprising the amino acid sequence of SEQ ID NO: 3. In one embodiment, the composition of the invention comprises a polynucleotide encoding a CdtB polypeptide comprising the amino acid sequence of SEQ ID NO: 4.

In one embodiment, the composition comprises a bacterium comprising at least one of PltA, PltB, PltC, CdtB, or a mutant thereof. In one embodiment, the composition comprises a bacterium comprising at least one nucleic acid encoding at least one of PltA, PltB, PltC, CdtB, or a mutant thereof. In one embodiment, the composition comprises a bacterium in which at least one of PltA, Pltb, PltC, CtdB, or a mutant thereof, are absent (i.e., toxin deficient). For example, in one embodiment, the composition comprises a S. typhi or a S. paratyphi bacterium.

In various embodiments, the invention provides a polypeptide, or a fragment of a polypeptide, a homolog, a mutant, a variant, a derivative or a salt of a polypeptide as elsewhere described herein, wherein the immunogenic activity of the polypeptide or fragment thereof is retained.

The invention should also be construed to include any form of a polypeptide having substantial homology to the polypeptides disclosed herein. In certain embodiments, a polypeptide which is “substantially homologous” is about 50% homologous, about 70% homologous, about 80% homologous, about 90% homologous, about 95% homologous, or about 99% homologous to amino acid sequence of the polypeptides disclosed herein.

In one embodiment, the polypeptide or combination of polypeptides of the present invention are capable of generating a specific immune response. In another embodiment, the polypeptide or combination of polypeptides of the present invention are capable of generating specific antibodies.

Polypeptides of the present invention can be prepared using well known techniques. For example, the polypeptides can be prepared synthetically, using either recombinant DNA technology or chemical synthesis. Polypeptides of the present invention may be synthesized individually or as longer polypeptides composed of two or more polypeptides. The polypeptides of the present invention can be isolated, i.e., substantially free of other naturally occurring host cell proteins and fragments thereof.

The polypeptides of the present invention may contain modifications, such as glycosylation, aglycosylation, side chain oxidation, or phosphorylation; so long as the modifications do not destroy the immunologic activity of the polypeptides. Other modifications include incorporation of D-amino acids or other amino acid mimetics that can be used, for example, to increase the serum half-life of the polypeptides.

The polypeptides of the invention can be modified whereby the amino acid is substituted for a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Note that the parenthetic letters indicate the one-letter codes of amino acids. As used herein, X stands for any amino acid.

The polypeptides of the invention can be prepared as a combination, which includes two or more of polypeptides of the invention, for use as a vaccine for prevention or treatment of S. typhi or S. paratyphi infection. The polypeptides may be in a cocktail or may be conjugated to each other using standard techniques. For example, the polypeptides can be expressed as a single polypeptide sequence. The polypeptides in the combination may be the same or different.

The present invention should also be construed to encompass “mutants,” “derivatives,” and “variants” of the polypeptides of the invention (or of the DNA encoding the same) which mutants, derivatives and variants are polypeptides which are altered in one or more amino acids (or, when referring to the nucleotide sequence encoding the same, are altered in one or more base pairs) such that the resulting polypeptide (or DNA) is not identical to the sequences recited herein, but has the same biological property as the polypeptides disclosed herein.

The nucleic acid sequences include both the DNA sequence that is transcribed into RNA and the RNA sequence that is translated into a polypeptide. According to other embodiments, the polynucleotides of the invention are inferred from the amino acid sequence of the polypeptides of the invention. As is known in the art several alternative polynucleotides are possible due to redundant codons, while retaining the biological activity of the translated polypeptides.

Further, the invention encompasses an isolated nucleic acid encoding a polypeptide having substantial homology to the polypeptides disclosed herein. In certain embodiments, the nucleotide sequence of an isolated nucleic acid encoding a polypeptide of the invention is “substantially homologous,” that is, is about 60% homologous, about 70% homologous, about 80% homologous, about 90% homologous, about 95% homologous, or about 99% homologous to a nucleotide sequence of an isolated nucleic acid encoding a polypeptide of the invention.

It is to be understood explicitly that the scope of the present invention encompasses homologs, analogs, variants, fragments, derivatives and salts, including shorter and longer polypeptides and polynucleotides, as well as polypeptide and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these modifications must preserve the immunologic activity of the original molecule. Specifically any active fragments of the active polypeptides as well as extensions, conjugates and mixtures are included and are disclosed herein according to the principles of the present invention.

The invention should be construed to include any and all isolated nucleic acids which are homologous to the nucleic acids described and referenced herein, provided these homologous nucleic acids encode polypeptides having the biological activity of the polypeptides disclosed herein.

The skilled artisan would understand that the nucleic acids of the invention encompass an RNA or a DNA sequence encoding a polypeptide of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

Further, any number of procedures may be used for the generation of mutant, derivative or variant forms of a protein of the invention using recombinant DNA methodology well known in the art such as, for example, that described in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). Procedures for the introduction of amino acid changes in a polypeptide or polypeptide by altering the DNA sequence encoding the polypeptide are well known in the art and are also described in these, and other, treatises.

### Vectors

The nucleic acids encoding the polypeptide or combinations of polypeptides of the invention of the invention can be incorporated into suitable vectors, including but not limited to, plasmids and retroviral vectors. Such vectors are well known in the art and are therefore not described in detail herein.

In one embodiment, the invention includes a nucleic acid sequence encoding one or more polypeptides of the invention operably linked to a nucleic acid comprising a promoter/regulatory sequence such that the nucleic acid is capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). The incorporation of a desired polynucleotide into a vector and the choice of vectors is well-known in the art as described in, for example, Sambrook et al. (2012), and in Ausubel et al. (1997).

The polynucleotide can be cloned into a number of types of vectors. However, the present invention should not be construed to be limited to any particular vector. Instead, the present invention should be construed to encompass a wide plethora of vectors which are readily available and/or well-known in the art. For example, the polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the desired nucleotide sequences of the invention, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or polypeptides. The promoter may be heterologous or endogenous.

One example of a constitutive promoter sequence is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter, where the promoter is active only in a desired tissue. Tissue-specific promoters are well known in the art and include, but are not limited to, the HER-2 promoter and the PSA associated promoter sequences.

In order to assess the expression of the nucleotide sequences encoding the polypeptide or combinations of polypeptides of the invention, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the expression vector is modified to increase the expression of the desired polypeptide. For example, the vector can undergo codon optimization to improve expression in a given mammal. For example, the vector can be codon-optimized for human expression. In another embodiment, the expression vector comprises an effective secretory leader. An exemplary leader is an IgE leader sequence. In another embodiment, the expression vector comprises a Kozak element to initiate translation. In another embodiment, the nucleic acid is removed of cis-acting sequence motifs/RNA secondary structures that would impede translation. Such modifications, and others, are known in the art for use in DNA vaccines (Kutzler et al, 2008, Nat. Rev. Gen. 9: 776-788; PCT App. No. PCT/US2007/000886; PCT App. No.; PCT/US2004/018962).

### Bacterium

In certain aspects, the compositions comprise a bacterium comprising at least one of PltA, PltB, PltC, CdtB, or a mutant thereof. In one embodiment, the composition comprises a bacterium comprising at least one nucleic acid molecules encoding at least one of PltA, PltB, PltC, CdtB, or a mutant thereof.

A bacterium comprising a nucleotide sequence encoding a mutant PltA, PltB, PltC, or CdtB polypeptide, can be generated using any method known in the art including, but not limited to allelic exchange and site-directed mutagenesis.

Any bacterium or bacterial strain which has at least one nucleotide sequence encoding one or more of PltA, PltB, PltC, CdtB, or a mutant thereof can be selected and used in accordance with the invention. In one embodiment, naturally occurring mutants or variants, or spontaneous mutants can be selected. In another embodiment, mutant bacteria can be generated by exposing the bacteria to mutagens, such as ultraviolet irradiation or chemical mutagens, or by multiple passages and/or passage in non-permissive hosts. Screening in a differential growth system can be used to select for those mutants having a mutation in at least one of PltA, PltB, PltC, or CdtB.

In another embodiment, mutations can be engineered into a bacterium, for example S. typhi or S. paratyphi bacterium using “reverse genetics” approaches. In this way, natural or other mutations which confer the inactivated toxin phenotype can be engineered into strains. For example, deletions, insertions or substitutions of the coding region of the gene responsible for the PltA, PltB, PltC, or CdtB proteins can be engineered. Deletions, substitutions or insertions in the non-coding region of the gene responsible for the PltA, PltB, PltC, or CdtB protein are also contemplated. To this end, mutations in the signals responsible for the transcription, replication, polyadenylation and/or packaging of the gene responsible PltA, PltB, PltC, or CdtB protein can be engineered.

In one embodiment, the bacterium is engineered to be toxin deficient, in which one or more of PltA, PltB, PltC, or CdtB is absent. For example, in certain embodiments, a toxin-deficient mutant bacterium or virus, where one or more of PltA, PltB, PltC, or CdtB is absent, is unable to cause disease but is able to induce an adaptive immune response against S. typhi or S. paratyphi.

Bacterium generated by the approaches described herein can be used in the vaccine and pharmaceutical formulations described herein. Reverse genetics techniques can also be used to engineer additional mutations to other genes important for vaccine production—i.e., the epitopes of useful vaccine strain variants can be engineered into the bacterium. Alternatively, completely foreign epitopes, including antigens derived from other pathogens can be engineered into the inactivated or attenuated strain.

The inactivated or attenuated bacterium of the present invention can itself be used as the active ingredient in vaccine or pharmaceutical formulations. In certain embodiments, the bacterium can be used as the vector or “backbone” of recombinantly produced vaccines. To this end, the “reverse genetics” technique can be used to engineer mutations or introduce foreign epitopes into the bacterium, which would serve as the “parental” strain. In this way, vaccines can be designed for immunization against strain variants, or in the alternative, against completely different infectious agents or disease antigens.

For example, in one embodiment, the immunological composition of the invention comprises a bacterium, engineered to express one or more epitopes or antigens of a given pathogen. For example, the bacterium can be engineered to express neutralizing epitopes of other preselected strains. Alternatively, epitopes of other pathogens can be built into the mutant bacterium.

In one embodiment, the bacterium is capable of inducing a robust immune response in the host—a feature which contributes to the generation of a strong immune response when used as a vaccine, and which has other biological consequences that make the bacterium useful as pharmaceutical agents for the prevention and/or treatment of an infection, disease, or disorder associated with an antigen. For example, in certain embodiments, the bacterium induces an anti-S. typhi or anti-S. paratyhpi immune response. In certain embodiments, the bacterium expressing one or more of PltA, PltB, PltC, CdtB, or a mutant thereof, serves as an adjuvant to enhance an immune response directed against a desired antigen.

### Antigen

The present invention provides compositions that induce an adaptive immune response (e.g., humoral immune response, cell-mediated immune response, etc.) in a subject. In one embodiment, the composition comprises an antigen. In one embodiment, the composition comprises a nucleic acid sequence which encodes an antigen. The antigen may be any molecule or compound, including but not limited to a polypeptide, peptide, protein, glycoprotein, saccharide, polysaccharide, or lipopolysaccharide, that induces an adaptive immune response in a subject.

In one embodiment, the antigen comprises a polypeptide or peptide associated with a pathogen, such that the antigen induces an adaptive immune response against the antigen, and therefore the pathogen. In one embodiment, the antigen comprises a fragment of a polypeptide or peptide associated with a pathogen, such that the antigen induces an adaptive immune response against the pathogen.

In certain embodiments, the antigen comprises an amino acid sequence that is substantially homologous to the amino acid sequence of an antigen described herein and retains the immunogenic function of the original amino acid sequence. For example, in certain embodiments, the amino acid sequence of the antigen has a degree of identity with respect to the original amino acid sequence of at least 60%, of at least 70%, of at least 85%, or of at least 95%.

In one embodiment, the antigen is encoded by a nucleic acid sequence of a nucleic acid molecule. In certain embodiments, the nucleic acid sequence comprises DNA, RNA, cDNA, a variant thereof, a fragment thereof, or a combination thereof. In certain instances, the nucleic acid sequence comprises include additional sequences that encode linker or tag sequences that are linked to the antigen by a peptide bond.

In certain embodiments, the antigen comprises a protein, peptide, polysaccharide, a fragment thereof, or a variant thereof, or a combination thereof from any number of organisms, for example, a virus, a parasite, a bacterium, a fungus, or a mammal.

In some embodiments, the antigen is a bacterial polysaccharide. Exemplary bacterial polysaccharide antigens include, but are not limited to, S. typhi Vi antigen and Streptococcus pneumoniae polysaccharide antigens.

In one embodiment, the antigen is an S. typhi or S. paratyphi antigen. Exemplary S. typhi or S. paratyphi antigens include, but are not limited to PltA, PltB, PltC, CdtB, and bacterial polysaccharide antigens, such as Vi antigen.

In certain embodiments, the antigen is associated with an autoimmune disease, allergy, or asthma. In other embodiments, the antigen is associated with cancer, herpes, influenza, hepatitis B, hepatitis C, human papilloma virus (HPV), ebola, pneumococcus, Haemophilus influenza, meningococcus, dengue, tuberculosis, malaria, norovirus or human immunodeficiency virus (HIV). In certain embodiments, the antigen comprises a consensus sequence based on the amino acid sequence of two or more different organisms. In certain embodiments, the nucleic acid sequence encoding the antigen is optimized for effective translation in the organism in which the composition is delivered.

In one embodiment, the antigen comprises a cancer antigen, tumor-specific antigen, or tumor-associated antigen, such that the antigen induces an adaptive immune response against the tumor. In one embodiment, the antigen comprises a fragment of a tumor-specific antigen or tumor-associated antigen, such that the antigen induces an adaptive immune response against the tumor. In certain embodiment, the tumor-specific antigen or tumor-associated antigen is a mutation variant of a host protein.

### Vaccine

For an antigenic composition to be useful as a vaccine, the antigenic composition must induce an immune response to the antigen in a cell, tissue or subject (e.g., a human). In certain aspects, the vaccine induces a protective immune response in the subject. As used herein, an “immunological composition” may comprise, by way of examples, an antigen (e.g., a polypeptide), a nucleic acid encoding an antigen (e.g., an antigen expression vector), or a cell expressing or presenting an antigen. In particular embodiments the antigenic composition comprises or encodes all or part of any polypeptide antigen described herein, or an immunologically functional equivalent thereof. In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

In the context of the present invention, the term “vaccine” (also referred to as an immunogenic composition) refers to a substance that induces immunity upon inoculation into an animal. In one embodiment, the vaccine induces anti-S. typhi or S. paratyphi immunity. In various embodiments, the vaccine of the invention comprises at least one of PltA, PltB, PltC, CdtB, or a mutant thereof, of S. typhi or and S. paratyphi to a subject to induce an immune response. In one embodiment, the vaccine is administered in combination with an adjuvant. In another embodiment, the vaccine is administered in the absence of an adjuvant.

In one embodiment, the vaccine comprises an antigen or nucleic acid encoding an antigen. For example, in one embodiment PltA, PltB, PltC, CdtB, or a mutant thereof of the vaccine or expressed by the vaccine, enhance an immune response directed against the chosen antigen. As described elsewhere herein, the antigen may be any suitable antigen to which an immune response is desired.

A vaccine of the present invention may vary in its composition of nucleic acid and/or cellular components. In a non-limiting example, a nucleic encoding an antigen might also be formulated with an adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

In one embodiment, the polypeptide vaccine of the invention includes, but is not limited to at least one polypeptide, or a fragment thereof, optionally mixed with adjuvant substances. In some embodiments, the polypeptide is introduced together with an antigen presenting cell (APC). The most common cells used for the latter type of vaccine are bone marrow and peripheral blood derived dendritic cells, as these cells express costimulatory molecules that help activation of T cells. WO00/06723 discloses a cellular vaccine composition which includes an APC presenting tumor associated antigen polypeptides. Presenting the polypeptide can be effected by loading the APC with a polynucleotide (e.g., DNA, RNA) encoding the polypeptide or loading the APC with the polypeptide itself.

Thus, the present invention also encompasses a method of inducing S. typhi or S. paratyphi immunity using one or more of polypeptides described herein. When a certain polypeptide or combination of polypeptides induces an S. typhi or S. paratyphi immune response upon inoculation into an animal, the polypeptide or combination of polypeptides are determined to have an immunity inducing effect. The induction of the S. typhi or S. paratyphi immunity by a polypeptide or combination of polypeptides can be detected by observing in vivo or in vitro the response of the immune system in the host against the polypeptide.

In one aspect, the present invention provides a method of enhancing an immune response directed against a desired antigen using one or more of the polypeptides described herein. When a certain polypeptide or combination of polypeptides enhances an antigen-specific immune response upon inoculation into an animal, the polypeptide or combination of polypeptides are determined to have a modulating immunogenic effect. The enhancement of an immune response by a polypeptide or combination of polypeptides can be detected by observing in vivo or in vitro the response of the immune system in the host against the desired antigen.

In another embodiment, the methods of the invention comprise administering to the subject a bacterium or virus comprising a nucleic acid sequence encoding at least one of PltA, PltB, PltC, CdtB, or a mutant thereof. In another embodiment, the methods of the invention comprise administering to the subject a bacterium or virus expressing at least a portion of at least one of PltA, PltB, PltC, CdtB, or a mutant thereof. In another embodiment, the methods of the invention comprise administering to the subject a bacterium or virus comprising at least a portion of at least one of PltA, PltB, PltC, CdtB, or a mutant thereof. In another embodiment, the methods of the invention comprise administering to the subject a bacterium or virus, wherein one or more of PltA, PltB, PltC, or CdtB is absent. For example, in certain embodiments, administering a toxin-deficient mutant bacterium or virus, where one or more of PltA, PltB, PltC, or CdtB is absent, is unable to cause disease but is able to induce an adaptive immune response against an antigen, including for example a S. typhi or S. paratyphi antigen.

For example, a method for detecting the induction of cytotoxic T lymphocytes is well known. A foreign substance that enters the living body is presented to T cells and B cells by the action of APCs. T cells that respond to the antigen presented by APC in an antigen-specific manner differentiate into cytotoxic T cells (also referred to as cytotoxic T lymphocytes or CTLs) due to stimulation by the antigen. These antigen stimulated cells then proliferate. This process is referred to herein as “activation” of T cells. Therefore, CTL induction by a certain polypeptide or combination of polypeptides of the invention can be evaluated by presenting the polypeptide to a T cell by APC, and detecting the induction of CTL. Furthermore, APCs have the effect of activating CD4+ T cells, CD8+ T cells, macrophages, eosinophils and NK cells.

A method for evaluating the inducing action of CTL using dendritic cells (DCs) as APC is well known in the art. DC is a representative APC having the strongest CTL inducing action among APCs. In this method, the polypeptide or combination of polypeptides are initially contacted with DC and then this DC is contacted with T cells. Detection of T cells having cytotoxic effects against the cells of interest after the contact with DC shows that the polypeptide or combination of polypeptides have an activity of inducing the cytotoxic T cells. Furthermore, the induced immune response can be also examined by measuring IFN-gamma produced and released by CTL in the presence of antigen-presenting cells that carry immobilized polypeptide or combination of polypeptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The polypeptide, or combination of polypeptides, confirmed to possess CTL inducing activity by these methods are polypeptides having DC activation effect and subsequent CTL inducing activity. Therefore, a polypeptide or combination of polypeptides that induce CTL against toxin A and toxin B are useful as vaccines against S. typhi or S. paratyphi associated pathology. Furthermore, CTL that have acquired cytotoxicity due to presentation of the polypeptide or combination of polypeptides by APC can be also used as vaccines against S. typhi or S. paratyphi infection.

Generally, when using a polypeptide for cellular immunotherapy, efficiency of the CTL-induction can be increased by combining a plurality of polypeptides having different structures and contacting them with DC. Therefore, when stimulating DC with protein fragments, it is advantageous to use a mixture of multiple types of fragments.

The induction of S. typhi or S. paratyphi immunity by a polypeptide or combination of polypeptides can be further confirmed by observing the induction of antibody production against the specific toxins. For example, when antibodies against a polypeptide or combination of polypeptides are induced in a laboratory animal immunized with the polypeptide or combination of polypeptides, and when S. typhi or S. paratyphi associated pathology is suppressed by those antibodies, the polypeptide or combination of polypeptides are determined to induce anti-S. typhi or anti-S. paratyphi immunity.

S. typhi or S. paratyphi immunity can be induced by administering a vaccine of the invention, and the induction of S. typhi or S. paratyphi immunity enables treatment and prevention of pathologies associated with S. typhi or S. paratyphi. Thus, the invention provides a method for treating, or preventing infection by S. typhi or S. paratyphi.

In certain aspects, enhancement of an antigen-specific immune response by one or more of PltA, PltB, PltC, CdtB, or a mutant thereof, enables treatment or prevention of an infection, disease or disorder associated with the desired antigen. Thus, the invention provides a method of treating or preventing an infection, disease or disorder associated with the desired antigen.

The therapeutic compounds or compositions of the invention may be administered prophylactically or therapeutically to subjects suffering from, or at risk of, or susceptible to, developing an infection, disease, or disorder associated with the antigen. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The polypeptide or combination of polypeptides of the invention having immunological activity, or a polynucleotide or vector encoding such a polypeptide or combination of polypeptides, may optionally be combined with an adjuvant. An adjuvant refers to a compound that enhances the immune response against the polypeptide or combination of polypeptides when administered together (or successively) with the polypeptide having immunological activity. Examples of suitable adjuvants include cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

### Administration

In one embodiment, the methods of the present invention comprise administering a composition comprising at least one polypeptide of the invention, and/or at least one polynucleotide encoding at least one polypeptide of the invention, to a subject. Administration of the composition can comprise, for example, intramuscular, intravenous, peritoneal, subcutaneous, intradermal, as well as topical administration.

In some embodiments, the at least one PltA, PltB, PltC, CdtB, or a mutant thereof, is linked to or conjugated to the antigen. In some embodiments, the at least one PltA, PltB, PltC, CdtB, or a mutant thereof, is not linked to or conjugated to the antigen, but is coadministered with the antigen.

The actual dose and schedule can vary depending on whether the compositions are administered in combination with other pharmaceutical compositions, or depending on inter-individual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Therapeutic Inhibitor Compositions and Methods

In various embodiments, the present invention includes inhibitor compositions for inhibiting the expression, activity, or both of S. typhi or S. paratyphi toxin. In one embodiment, the inhibitor compositions inhibit the expression, activity, or both of one or more of PltA, PltB, PltC, CdtB, or a mutant thereof. In certain embodiments, the inhibitor compositions inhibit the interaction between the S. typhi or S. paratyphi toxin and the toxin's receptor. Inhibition of the interaction between the S. typhi or S. paratyphi toxin and the toxin's receptor can be assessed using a wide variety of methods, including those disclosed herein, as well as methods known in the art or to be developed in the future.

One skilled in the art, based upon the disclosure provided herein, would understand that the inhibitor compositions and methods of the invention are useful in treating preventing and infection by S. typhi and S. paratyphi.

The inhibitor compositions and methods of the invention include, but should not be construed as being limited to, a chemical compound, a protein, a peptide, a peptidomemetic, an antibody, a ribozyme, a small molecule chemical compound, a glycan, an antisense nucleic acid molecule (e.g., siRNA, miRNA, etc.), or combinations thereof.

One of skill in the art would readily appreciate, based on the disclosure provided herein, that the inhibitor compositions of the invention include those that interfere with the interaction between the toxin and its receptor. In some embodiments, the inhibitor compositions bind to the toxin and interfere with the interaction between the toxin and its receptor. In other embodiments, the inhibitor compositions bind to the toxin's receptor and interfere with the interaction between the toxin and its receptor.

In various embodiments, the treatment of S. typhi or S. paratyphi infection in a subject is accomplished through passive antibody therapy (i.e., the transfer of antibodies to the S. typhi or S. paratyphi infected subject). In various embodiments, the inhibitor compositions and methods of the invention are used in combination with an antibiotic therapy. When used in combination, the antibiotic therapy can be administered before, during or after the administration of the inhibitor compositions of the invention.

In some embodiments, the receptor for the S. typhi or S. paratyphi toxin is a glycan. In one embodiment, the glycan is an N-linked glycan such as, by way of non-limiting examples, a sialylated tri- or bi-antennary glycan with one or all of the branches terminally sialyated. In another embodiment, the glycan is a non-sialylated tri- or bi-antennary glycan. In another embodiment, the glycan is a ganglioside. In another embodiment, the glycan is a glycan found on O-glycans.

In certain embodiments, the inhibitor compositions comprise an antibody or antibody fragment. For example, in one embodiment, the inhibitor compositions comprise an antibody or antibody fragment that specifically binds to S. typhi toxin, S. paratyphi toxin, S. typhi toxin receptor, or S. paratyphi toxin receptor. In one embodiment, the antibody or antibody fragment specifically binds to one or more of PltA, PltB, PltC, CdtB, or a mutant thereof. In certain embodiment the antibody or antibody fragment inhibits the activity of one or more components of S. typhi toxin or S. paratyphi toxin, including but not limited to one or more of PltA, PltB, PltC, CdtB, or a mutant thereof.

Methods of making and using antibodies are well known in the art. For example, polyclonal antibodies useful in the present invention can be generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magenetic-actived cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, at least about 80%, at least about 90%, at least about 95%, or at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Further, one of skill in the art would, when equipped with this disclosure and the methods exemplified herein, appreciate that an inhibitor composition includes such inhibitors as discovered in the future, as can be identified by well-known criteria in the art of pharmacology, such as the physiological results of inhibition of the toxin as described in detail herein and/or as known in the art. Therefore, the present invention is not limited in any way to any particular inhibitor composition as exemplified or disclosed herein; rather, the invention encompasses those inhibitor compositions that would be understood by the routineer to be useful as are known in the art and as are discovered in the future.

Further methods of identifying and producing inhibitor compositions are well known to those of ordinary skill in the art, including, but not limited, obtaining an inhibitor from a naturally occurring source (i.e., Streptomyces sp., Pseudomonas sp., Stylotella aurantium). Alternatively, an inhibitor can be synthesized chemically. Further, the routineer would appreciate, based upon the teachings provided herein, that an inhibitor composition can be obtained from a recombinant organism. Compositions and methods for chemically synthesizing inhibitors and for obtaining them from natural sources are well known in the art and are described in the art.

One of skill in the art will appreciate that an inhibitor can be administered as a small molecule chemical, a protein, an antibody, a glycan, a nucleic acid construct encoding a protein, an antisense nucleic acid, a nucleic acid construct encoding an antisense nucleic acid, or combinations thereof. In one embodiment, the inhibitor composition of the invention that interferes with the interaction between the S. typhi or S. paratyphi toxin and the toxin's receptor is a soluble form of at least a fragment of at least one glycan that is a receptor for the S. typhi or S. paratyphi toxin.

Numerous vectors and other compositions and methods are well known for administering a protein or a nucleic acid construct encoding a protein to cells or tissues. Therefore, the invention includes a method of administering a protein or a nucleic acid encoding a protein that is an inhibitor. (Sambrook et al., 2012, Molecular Cloning: A Laboratory Manual, Cold Spring Harbor Laboratory, New York; Ausubel et al., 1997, Current Protocols in Molecular Biology, John Wiley & Sons, New York).

One of skill in the art will appreciate that inhibitors of the invention can be administered singly or in any combination. Further, inhibitors can be administered singly or in any combination in a temporal sense, in that they may be administered concurrently, or before, and/or after each other.

In various embodiments, any of the inhibitors of the invention described herein can be administered alone or in combination with other inhibitors of other molecules associated with S. typhi or S. paratyphi infection.

It will be appreciated by one of skill in the art, when armed with the present disclosure including the methods detailed herein, that the invention is not limited to treatment of a disease or disorder that is already established. Particularly, the disease or disorder need not have manifested to the point of detriment to the subject; indeed, the disease or disorder need not be detected in a subject before treatment is administered. That is, significant disease or disorder does not have to occur before the present invention may provide benefit. Therefore, the present invention includes a method for preventing infection in a subject, in that an inhibitor composition, as discussed previously elsewhere herein, can be administered to a subject prior to the onset of the infection.

The invention encompasses administration of an inhibitor to practice the methods of the invention; the skilled artisan would understand, based on the disclosure provided herein, how to formulate and administer the appropriate inhibitor to a subject. Indeed, the successful administration of the inhibitor has been reduced to practice herein. However, the present invention is not limited to any particular method of administration or treatment regimen.

### Pharmaceutical Compositions

The present invention includes the treatment of a S. typhi or S. paratyphi infection in a subject by the administration of a therapeutic composition of the invention to a subject in need thereof. In one embodiment, the therapeutic composition of the invention is an inhibitor composition. In one embodiment, the therapeutic composition of the invention for the treatment of S. typhi or S. paratyphi infection is at least one antibody that specifically binds to at least one of PltA, PltB, PltC, CdtB, or a mutant thereof. In various embodiments, the treatment of S. typhi or S. paratyphi infection in a subject is accomplished through passive antibody therapy (i.e., the transfer of antibodies to the S. typhi or S. paratyphi infected subject).

Administration of the therapeutic composition in accordance with the present invention may be continuous or intermittent, depending, for example, upon the recipient's physiological condition, whether the purpose of the administration is therapeutic or prophylactic, and other factors known to skilled practitioners. The administration of the compositions of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

In some embodiments, when the compositions of the invention are prepared for administration, they are combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the compositions of the invention can be prepared by procedures known in the art using well known and readily available ingredients. The compositions of the invention can also be formulated as solutions appropriate for parenteral administration, for instance by intramuscular, subcutaneous or intravenous routes.

Thus, the composition may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Methods of Diagnosis

In other embodiments, the invention is a method of determining whether a subject is, or has been, infected with S. typhi or S. paratyphi. In some embodiments, the method comprises detecting or measuring the level of typhoid toxin in the subject. In various embodiments, the method comprises detecting or measuring the level of at least one of PltA, CdtB, PltC, and PltB in the subject. In some embodiments, the method comprises detecting or measuring the level of antibodies that specifically bind to the typhoid toxin in the subject. In various embodiments, the method comprises detecting or measuring the level of at least one antibody that specifically binds to PltA, PltC, CdtB or PltB in the subject.

In one embodiment, the invention is a method of determining whether a subject is infected with S. typhi or S. paratyphi, comprising the step of detecting or measuring the level of typhoid toxin in a biological sample of the subject. In various embodiments, the method comprises detecting or measuring the level of typhoid toxin by detecting or measuring the level of at least one of PltA, PltC, CdtB and PltB in the biological sample of the subject. In various embodiments, to determine whether the level of typhoid toxin is elevated in a biological sample of the subject, the level of typhoid toxin is compared with the level of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject.

In one embodiment, the invention is a method of determining whether a subject is, or has been, infected with S. typhi or S. paratyphi, comprising the step of detecting or measuring the level of antibodies that specifically bind to the typhoid toxin in a biological sample of the subject. In various embodiments, the method comprises detecting or measuring the level of antibodies that specifically bind to typhoid toxin by detecting or measuring the level of at least one antibody that specifically binds to PltA, PltC, CdtB, or PltB in the biological sample of the subject. In various embodiments, to determine whether the level of antibodies that specifically bind to typhoid toxin is elevated in a biological sample of the subject, the level of antibodies that specifically bind to typhoid toxin is compared with the level of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject.

The present invention also includes determining whether a subject is, or has been, infected with S. typhi or S. paratyphi by detecting one or more biomarkers associated with S. typhi or S. paratyphi toxin activity. Exemplary biomarkers include, but are not limited to, DNA, RNA, and protein biomarkers. In certain embodiments, the biomarkers are one or more of DNA, RNA, and protein biomarkers of the host, where the level of each biomarker being either increased or decreased, as compared to a control subject, is indicative of S. typhi or S. paratyphi infection. In certain embodiments, the biomarkers are one or more of DNA, RNA, and protein biomarkers of S. typhi or S. paratyphi, where the level of each biomarker being either increased or decreased, as compared to a control subject, is indicative of S. typhi or S. paratyphi infection. In one embodiment, the invention is a method of determining whether a subject is, or has been, infected with S. typhi or S. paratyphi, comprising the step of detecting or measuring the level of one or more biomarkers associated with S. typhi or S. paratyphi toxin activity in a biological sample of the subject. In various embodiments, the method comprises detecting or measuring the level of one or more biomarkers associated with S. typhi or S. paratyphi toxin activity by detecting or measuring the level of at least one biomarker associated with S. typhi or S. paratyphi toxin activity in the biological sample of the subject. In various embodiments, to determine whether the level of one or more biomarkers associated with S. typhi or S. paratyphi toxin activity is elevated or diminished in a biological sample of the subject, the level of one or more biomarkers associated with S. typhi or S. paratyphi toxin activity is compared with the level of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject.

In various embodiments of the methods of the invention, the level of at least one of PltA, PltC, CdtB, PltB, an antibody the specifically binds to PltA, an antibody that specifically binds to PltC, an antibody the specifically binds to CdtB, an antibody the specifically binds to PltB levels, or one or more biomarkers associated with S. typhi or S. paratyphi toxin activity is determined to be elevated when the level of antibody that specifically binds to typhoid toxin is increased by at least 1%, at least 5%, at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In various embodiments of the methods of the invention, the level of at least one of PltA, PltC, CdtB, PltB, an antibody the specifically binds to PltA, an antibody that specifically binds to PltC, an antibody the specifically binds to CdtB, an antibody the specifically binds to PltB levels, or one or more biomarkers associated with S. typhi or S. paratyphi toxin activity is determined to be diminished when the level of antibody that specifically binds to typhoid toxin is decreased by at least 1%, at least 5%, at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In various embodiments, the biological sample is a sample containing a polypeptide or nucleic acid of at least one of PltA, PltC, CdtB, PltB, an antibody the specifically binds to PltA, an antibody that specifically binds to PltC, an antibody the specifically binds to CdtB, an antibody the specifically binds to PltB, or one or more biomarkers associated with S. typhi or S. paratyphi toxin activity. The biological sample can be a sample from any source which contains a polypeptide or a nucleic acid, such as a bodily fluid or a tissue, or a combination thereof. A biological sample can be obtained by appropriate methods, such as, by way of examples, blood draw, fluid draw, or biopsy. A biological sample can be used as the test sample; alternatively, a biological sample can be processed to enhance access to the polypeptides or nucleic acids, or copies of the nucleic acids, and the processed biological sample can then be used as a test sample.

In various embodiments of the invention, methods of detecting or measuring the level of at least one of PltA, PltC, CdtB, PltB, an antibody the specifically binds to PltA, an antibody that specifically binds to PltC, an antibody the specifically binds to CdtB, an antibody the specifically binds to PltB levels, or one or more biomarkers associated with S. typhi or S. paratyphi toxin activity in a biological sample obtained from a patient include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (MA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In various embodiments of the invention, methods of detecting or measuring the level of at least one of PltA, PltC, CdtB, PltB, an antibody the specifically binds to PltA, an antibody that specifically bind to PltC, an antibody the specifically binds to CdtB, an antibody the specifically binds to PltB levels, or one or more biomarkers associated with S. typhi or S. paratyphi toxin activity in a biological sample obtained from a patient include, but are not limited to, quantitative hybridization methods, such as Southern analysis, Northern analysis, or in situ hybridizations, (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA or cDNA. The hybridization sample is maintained under conditions which are sufficient to allow specific hybridization of the nucleic acid probe to mRNA or cDNA. Specific hybridization can be performed under high stringency conditions or moderate stringency conditions, as appropriate. In one embodiment, the hybridization conditions for specific hybridization are high stringency. Specific hybridization, if present, is then detected using standard methods. If specific hybridization occurs between the nucleic acid probe having a mRNA or cDNA in the test sample, the level of the mRNA or cDNA in the sample can be assessed. More than one nucleic acid probe can also be used concurrently in this method. Specific hybridization of any one of the nucleic acid probes is indicative of the presence of the mRNA or cDNA of interest, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the biological sample.

In another embodiment, arrays of oligonucleotide probes that are complementary to target nucleic acid sequences in the biological sample obtained from a subject can be used to determine the level of one or more biomarkers, including typhoid toxin in the biological sample of a subject. The array of oligonucleotide probes can be used to determine the level of one or more biomarkers, including typhoid toxin, or at least one of PltA, PltC, CdtB, PltB, alone, or in relation to the level of one or more other nucleic acids in the biological sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparator controls (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of 32P, 33P, 35S or 3H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a biological sample, including a biological fluid and fresh or fixed tissue sample.

There are many methods known in the art for the detection and quantification of specific nucleic acid sequences and new methods are continually reported. A great majority of the known specific nucleic acid detection and quantification methods utilize nucleic acid probes in specific hybridization reactions. In one embodiment, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In some aspects, in the Southern blot, the nucleic acid probe is labeled with a tag. That tag can be a radioactive isotope, a fluorescent dye or the other well-known materials. Another type of process for the specific detection of nucleic acids in a biological sample known in the art are the hybridization methods as exemplified by U.S. Pat. Nos. 6,159,693 and 6,270,974, and related patents. To briefly summarize one of those methods, a nucleic acid probe of at least 10 nucleotides, at least 15 nucleotides, or at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparator control nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. In one embodiment, the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In one embodiment, the process for determining the quantitative and qualitative profile of the nucleic acid of interest according to the present invention is characterized in that the amplifications are real-time amplifications performed using a labeled probe, such as a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. In one embodiment, the Tm for any one of the hydrolysis-probes of the present invention is in the range of about 67° C. to about 70° C. In one embodiment, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. In one embodiment, a primer featured in the invention includes a nucleotide sequence sufficiently complementary to hybridize to a nucleic acid sequence of about 12 to 25 nucleotides. In one embodiment, the primer differs by no more than 1, 2, or 3 nucleotides from the target flanking nucleotide sequence In another aspect, the length of the primer can vary in length, in certain embodiments about 15 to 28 nucleotides in length (e.g., 16, 17, 18, 19, 20, 21, 22, 23, 24, 25, 26, or 27 nucleotides in length).

In one aspect, the invention includes a method of preparing a sample comprising: a.) providing a sample of a subject, wherein the subject is suspected of having an S. typhi or S. paratyphi infection or the subject has a symptom of an S. typhi or S. paratyphi infection; b.) selectively extracting PltC, nucleic acid sequences encoding PltC, anti-PltC antibodies, or nucleic acid sequences encoding anti-PltC antibodies from the sample; and c.) performing an assay on the extracted PltC, nucleic acid sequences encoding PltC, anti-PltC antibodies, or nucleic acid sequences encoding anti-PltC antibodies in order to quantitatively or qualitatively detect the level of PltC, nucleic acid sequences encoding PltC, anti-PltC antibodies, or nucleic acid sequences encoding anti-PltC antibodies in the sample.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Alternate Subunit Assembly Diversifies the Function of a Bacterial Toxin

Bacterial toxins with an AB₅ architecture consist of an active (A) subunit inserted into a ring-like platform comprised of five delivery (B) subunits. Salmonella typhi, the cause of typhoid fever, produces an unusual A₂B₅ toxin known as typhoid toxin. Here, it is reported that upon infection of human cells, S. typhi produces two forms of typhoid toxin that have distinct delivery components but share common active subunits. The two typhoid toxins exhibit different trafficking properties, elicit different effects when administered to laboratory animals, and are expressed using different regulatory mechanisms and in response to distinct metabolic cues. Collectively, these results indicate that the evolution of two typhoid toxin variants has conferred functional versatility to this virulence factor. More broadly, this study reveals a new paradigm in toxin biology and suggests that the evolutionary expansion of AB₅ toxins was likely fueled by the plasticity inherent to their structural design coupled to the functional versatility afforded by the combination of homologous toxin components.

AB₅ toxins are a structurally similar but functionally diverse class of virulence factors that are widespread in bacteria. They play an important and well-documented role in the pathogenesis of many pathogens of great public health importance including Bordetella pertussis, E. coli, Vibrio cholerae, Shigella spp. and Salmonella spp (Beddoe et al., Trends Biochem. Sci. 35, 411-418 (2010); Merritt et al., Curr. Opin. Struct. Biol. 5, 165-171 (1995)). Their name is derived from their architectural organization, which consists of a pentameric “B” subunit, which targets the toxin to specific cells, and an associated catalytic “A” subunit, which is responsible for the harmful effects on cellular targets. Salmonella enterica serovar Typhi (S. typhi) is the etiological agent of typhoid fever, a major global health problem (Dougan et al., Annu Rev. Microbiol. 68, 317-336 (2014); Parry et al., N. Engl. J. Med. 347, 1770-1782 (2002)). An assortment of evidence indicates that typhoid toxin is responsible for some of the more severe symptoms of typhoid fever (Spano et al., Cell Host Microbe 3, 30-38 (2008); Song et al., Nature 499, 350-354 (2013); Galan, Proc. Natl Acad. Sci. USA 113, 6338-6344 (2016)). Compared to other AB-type toxins, typhoid toxin is highly unusual in that two A subunits, CdtB, a DNAse, and PltA, an ADP-ribosyltransferase, associate with a single pentameric B subunit, PltB, resulting in a unique A₂B₅ architecture (Song et al., Nature 499, 350-354 (2013)). This unusual composition appears to be the result of typhoid toxin's remarkable evolutionary history, during which two classes of AB-type toxins—cytolethal distending toxins (CDTs) and pertussis-like toxins—amalgamated to produce a single toxin (Gao et al., Nat. Microbiol. 2, 1592-1599 (2017)). A structural and biochemical investigation into the evolution of typhoid toxin revealed that this class of toxins exhibits remarkable plasticity in that heterologous co-expression of various combinations of homologs from other bacteria produced active typhoid-toxin-like complexes (Gao et al., Nat. Microbiol. 2, 1592-1599 (2017)). These observations raise the question whether alternative forms of AB₅ toxins could be assembled from homologous subunits encoded by the same bacterium.

Here it is shown that in S. typhi this is the case and that an alternative form of typhoid toxin is assembled involving a B subunit homolog encoded elsewhere in its chromosome. It is shown that the alternative form of typhoid toxin exhibits different biological properties and that the expression of its B subunit is controlled by a different regulatory network in response to different metabolic cues. These results have important implications for the biology of typhoid toxin and AB₅ toxins in general.

The materials and methods employed in these experiments are now described.

Bacterial Strains and Cell Lines

S. typhi strains employed in this study were derived from the wild-type isolate ISP2825 (Galan et al., Immun. 59, 2901-2908 (1991)) and were constructed using standard recombinant DNA and allelic exchange procedures using the E. coli β-2163 Δnic35 as the conjugative donor strain (Demarre et al., Res. Microbiol. 156, 245-255 (2005)). All the S. typhi deletion mutant strains carry deletions of the entire coding regions of the indicated gene or genes from the start to the stop codons. The malE-3×FLAG strain was generated by moving a C-terminal 3×FLAG tagged version of malE to the pltC locus (resulting in a pltC deletion). All other strains featuring 3×FLAG epitope-tagged genes were generated by replacing the native gene with a C-terminal 3×FLAG tagged version at its native genomics locus. A complete list of bacterial strains used in this study is provided in Table 3. Strains were routinely cultured in LB broth at 37° C. For in vitro growth assays that employed typhoid toxin inducing growth conditions (TTIM) a previously described chemically defined growth medium was used (Fowler et al., Cell Host Microbe 23, 65-76 (2018)) that was based on N minimal medium (Snavely et al., J. Biol. Chem. 266, 815-823 (1991)). All experiments using cultured cells were conducted using the Henle-407 human epithelial cell line, which was obtained from the Roy Curtiss library collection. Cells were cultured in Dulbecco's modified Eagle medium (DMEM, GIBCO) supplemented with 10% Fetal Bovine Serum (FBS) at 37° C. with 5% CO₂ in a humidified incubator. This cell line was routinely tested for mycoplasma contamination using a Mycoplasma Detection Kit (SouthernBiotech, Cat #13100-01).

Salmonella typhi Infections

To infect Henle-407 cells, overnight cultures of S. typhi were diluted 1/20 into fresh LB containing 0.3 M NaCl and grown to an OD₆₀₀ of 0.9. Cells were infected for 1 h in Hank's balanced salt solution (HBSS) at the indicated multiplicity of infection (MOI). Cells were then washed three times with HBSS and incubated in culture medium containing 100 μg/ml gentamycin to kill extracellular bacteria. After 1 h, cells were washed and fresh medium was added containing 5 μg/ml gentamycin to avoid repeated cycles of reinfection.

β-galactosidase Assays

For in vitro grown samples, overnight cultures were washed two times with TTIM, diluted 1/20 into fresh TTIM and grown for the indicated amount of time at 37° C. at which point 10 or 20 μl of the culture was added to 90 μl of permeabilization buffer (100 mM Na₂HPO₄, 20 mM KCl, 2 mM MgSO₄, 0.8 mg/ml hexadecyltrimethylammonium bromide [CTAB], 0.4 mg/ml sodium deoxycholate, 5.4 μL/ml β-mercaptoethanol) and assayed, as described below. For samples collected from infected cells, 3×10⁵ Henle-407 cells were plated in 6-well plates and grown for 24 h prior to infection with the indicated strains. Following infections, cells were washed two times with PBS, released from the plates using dilute trypsin and pelleted by centrifugation for 5 min at 150×g. Cells were then lysed in 0.1% sodium deoxycholate (in PBS) supplemented with 100 μg/ml DNase I and the lysate was centrifuged at 5000×g for 5 min to pellet the bacteria. The bacteria were re-suspended in PBS, a small aliquot of which was diluted to calculate the total number CFU recovered. The remainder of the bacteria were pelleted, re-suspended in 100 μl of permeabilization buffer and assayed as described below. Assays were conducted at 24 h post infection (hpi) unless otherwise indicated. β-galactosidase assays were conducted using a modified version of the protocol developed by Miller (Fowler et al., Cell Host Microbe 23, 65-76 (2018); Miller, J. H. Experiments in molecular genetics. (Cold Spring Harbor Laboratory, 1972)). Briefly, samples were permeabilized for 20 min at room temperature in the buffer described above after which 600 μl of substrate buffer (60 mM Na₂HPO₄, 40 mM NaH₂PO₄, 2.7 μL/mL β-mercaptoethanol, 1 mg/ml ONPG [o-nitrophenyl-β-D-galactopyranoside, Sigma]) was then added to initiate the reactions. Once the samples developed an obvious yellow color, the reactions were quenched using 700 μl of 1 M Na₂CO₃ and the reaction time was noted. Cell debris was removed by centrifugation at 20,000×g for 5 min and the OD₄₂₀ of the samples was measured. Miller units were calculated as: (1000×OD₄₂₀)/(reaction time [minutes]×culture volume assayed [ml]×OD₆₀₀ [culture]).

Co-Immunoprecipitation Experiments

To identify interaction partners for the various typhoid toxin subunits, S. typhi bacterial cell lysates were immunoprecipitated using C-terminal 3×FLAG epitope-tagged CdtB or PltC (tags were incorporated at the native genomic locus) as indicated and the eluates were analyzed by LC-MS/MS or western blot. A strain encoding a C-terminal 3×FLAG epitope-tagged version of MalE, a periplasmic protein that is expressed in TTIM, which was cloned in place of pltC at the pltC locus in the S. typhi chromosome and included as a negative control for the LC-MS/MS analysis. For in vitro grown samples, the indicated strains were grown overnight in LB, washed twice using TTIM, diluted 1/20 into 12 ml of fresh TTIM and grown overnight. Cultures were pelleted, re-suspended in lysis buffer (50 mM Tris pH 7.5, 170 mM NaCl, cOmplete mini protease inhibitors [Sigma], 40 ug/ml DNase I) and lysed using the One Shot cell disruption system (Constant Systems, Ltd). Clarified lysates were immunoprecipitated overnight at 4° C. using ANTI-FLAG M2 affinity gel (Sigma). Immunoprecipitated samples were washed thoroughly using 50 mM Tris pH 7.5/170 mM NaCl/50 mM galactose/0.1% Triton X-100 and eluted using 0.1 M glycine-HCl (LC-MS/MS) or SDS-PAGE loading buffer (western blot analysis). For LC-MS/MS analysis, the eluted samples were precipitated overnight at −20° C. in 80% acetone and washed twice using 80% acetone. The samples were then reduced using DTT, alkylated using iodoacetamide and trypsin digested overnight. C18 column purified peptides were then analyzed by LC/MS/MS, as previously described (Spanò et al., Cell Host Microbe 19, 216-226 (2016)) and MS/MS scans were processed and searched using MASCOT (Matrix Science Ltd.). The resulting peptide and protein assignments were filtered to keep only those identifications with scores above extensive homology. For western blot analysis, eluted samples were run on 10-15% SDS-PAGE and transferred to nitrocellulose membranes. Membranes were blocked using 5% non-fat milk, incubated overnight at 4° C. with the indicated primary antibody followed by a 2 h incubation with a fluorescently conjugated α-mouse (FLAG) or α-rabbit (PltB) secondary antibody and analyzed using the Li-Cor Odyssey blot imager. The following antibodies were used for western blot analysis: α-FLAG mouse monoclonal (Sigma, F3165, 1:5000 dilution), α-PltB rabbit polyclonal (produced for Galan lab by Pocono Rabbit Farm & Laboratory, Inc. Canadensis Pa., 1:5000 dilution), α-CdtB rabbit polyclonal (produced for Galan lab by Pocono Rabbit Farm & Laboratory, 1:5000 dilution).

For samples isolated from infected cells, three 15 cm dishes containing a total of 7.5×10⁷ Henle-407 cells were infected at an MOI of 20 as described above using each of the indicated S. typhi strains. 24 h post-infection, cells were washed twice using PBS and collected using a cell scraper, pelleted and washed once using PBS. Cell pellets were then resuspended in lysis buffer (50 mM Tris pH 7.5, 170 mM NaCl, complete mini protease inhibitors [Sigma], 40 ug/ml DNase I, 10 mM N-ethylmaleimide) and lysed using a Branson Digital Sonifier (3 s on/8 s off, 35% amplitude, 3 min total). Clarified lysates were then immunoprecipitated and analyzed by western blot as described above.

Typhoid Toxin Purification

Both typhoid toxins were purified according to a previously established protocol (Song et al., Nature 499, 350-354 (2013)). Briefly, pltB, pltA and cdtB-6×His (PltB-typhoid toxin) or pltC, pltA and cdtB-6×His (PltC-typhoid toxin) were cloned into pET28a(+) vector (Novagen). E. coli strains carrying these expression vectors were grown to OD₆₀₀˜0.8, at which time 250 μM IPTG was added to induce the expression of the toxin genes and the cultures were grown overnight at 30° C. Bacterial cells were pelleted by centrifugation and lysed. Crude lysates were affinity purified using Nickel resin (Qiagen), followed by cation exchange chromatography using a Mono S column (Sigma-Aldrich) and finally gel filtration using a Superdex-200 column (Sigma-Aldrich). The final fractions were analyzed by SDS-PAGE to confirm purity.

Culture Cell Intoxication Assays

To assess typhoid toxin toxicity in cultured human cells, the number of cells arrested in G2/M (as a consequence of CdtB-mediated DNA damage) was determined using flow cytometry as previously described (Song et al., Nature 499, 350-354 (2013)). For experiments using purified toxin, 2.5×10⁴ Henle-407 epithelial cells were plated in 12-well plates. After 24 h the cells were washed and fresh medium containing the indicated concentrations of PltB- or PltC-typhoid toxin were added. Forty eight hours later, the cells were removed from the dishes using trypsin treatment, pelleted, re-suspended in 300 μl PBS, fixed by adding ice cold ethanol dropwise to a final concentration of 70% and incubated overnight at −20° C. Cells were then washed with PBS, re-suspended in 500 μl of PBS containing 50 μg/ml propidium iodide, 0.1 μg/ml RNase A and 0.05% Triton X-100 and incubated for 30 min at 37° C. Cells were then washed and re-suspended in PBS, filtered and analyzed by a flow cytometry. The DNA content of cells was determined using FlowJo (Treestar). In order to obtain EC₅₀ values, the percentage of cells in the G2/M phase (% G2/M) was determined for each sample and converted to normalized toxicity by subtracting the % G2-M value observed of untreated cells and dividing by (maximum G2/M−untreated % G2/M). The maximum % G2/M value was considered to be 90% based on experience that values above this number are not reliably observed even at saturating toxin concentrations. For experiments using S. typhi infected cells, 2.5×10⁴ Henle-407 cells were plated in 12-well plates and were infected using the indicated S. typhi strains at the indicated MOI values 24 h later. After 48 h post-infection the cells were collected, fixed, stained and analyzed as described above.

Typhoid Toxin Secretion Assay

To assess the mechanism of secretion for the PltB- and PltC typhoid toxins, an in vitro toxin secretion assay was employed to determine whether toxin secretion from ΔpltB (producing exclusively PltC-typhoid toxin) and ΔpltC (producing exclusively PltB-typhoid toxin) S. typhi strains was dependent upon both TtsA and outer membrane perturbation, in accordance with the recently described mechanism of typhoid toxin secretion (Geiger et al., Nat. Microbiol. 3, 1243-1254 (2018)). The indicated strains, all of which encode 3×-FLAG epitope-tagged cdtB from its native genomic locus, were grown overnight in LB, washed twice using TTIM, diluted 1/20 into fresh TTIM and grown for 24 h at 37° C. to induce typhoid toxin and ttsA expression. The bacteria were then pelleted, washed thoroughly, and incubated for 15 min either in 0.075% bile salts (Sigma) in PBS or in PBS alone (mock treated). The bacteria were then pelleted and the supernatants were filtered using a 0.2 μm filter and TCA precipitated. The amount of toxin in the pellet and supernatant fractions was then determined by western blot analysis using an M2 α-FLAG antibody as described above.

Immunofluorescence Microscopy Assay for Typhoid Toxin Export

To compare the export of typhoid toxin from the Salmonella-containing vacuole to the extracellular space for PltB-typhoid toxin and PltC-typhoid toxin, a previously established immunofluorescence-based assay was employed. The assay quantifies the levels typhoid toxin that are within exocytic vesicles compared to the levels associated with bacteria (Chang et al., Cell Host Microbe 20, 682-689 (2016)). Henle-407 cells plated on glass coverslips were infected with the indicated cdtB-3×FLAG epitope-tagged S. typhi strains at an MOI of 10. Forty-eight hours post-infection, the samples were fixed in 4% paraformaldehyde and blocked using 3% BSA/0.3% triton X-100 in PBS. The coverslips were then incubated with a 1:5000 dilution of mouse monoclonal M2 anti-FLAG antibody (Sigma) and a 1:10,000 dilution of rabbit polyclonal anti-S. typhi LPS antibody (Sifin) overnight at 4° C. After thoroughly washing in PBS, samples were then stained using Alexa 488-conjugated anti-mouse and Alexa 594-conjugated anti-rabbit antibodies and DAPI (Sigma) for 2 h at room temperature, washed extensively using PBS, mounted on coverslips, and imaged using an Eclipse TE2000 inverted microscope (Nikon) with an Andor Zyla 5.5 sCMOS camera driven by Micromanager software.

The open source software ImageJ was used to quantify toxin export in images captured in random fields as described previously (Chang et al., Cell Host Microbe 20, 682-689 (2016)). Briefly, the LPS signal was used to identify the bacterial cells and the CdtB-3×FLAG signal was used to identify typhoid toxin. The typhoid toxin signal within the area associated with bacterial cells was subtracted from the total typhoid toxin signal in order to obtain the signal associated with typhoid toxin carrier intermediates. In a given field, the fluorescence intensity of the typhoid toxin signal that was associated with toxin carriers was normalized to the bacterial-associated typhoid toxin signal within the same field.

Animal Intoxication Experiments

C57BL/6 mice were anesthetized with 30% w/v isoflurane in propylene glycol and 100 μl of toxin solution containing the indicated concentration of purified PltB- or PltC-typhoid toxin was administered via the retro-orbital route. Changes in behavior, weight and survival of the toxin-injected mice were closely monitored for the duration of the experiment. Blood samples were collected by cardiac puncture 4 days after toxin administration in Microtainer tubes coated with EDTA, kept at room temperature and analyzed within 2 h after blood collection using a HESKA Veterinary Hematology System.

FAST-INSeq Screen

The FAST-INSeq screen was employed to identify S. typhi genes that, when disrupted by transposon mutagenesis, lead to altered expression of a pltC:gfp reporter within infected Henle-407 epithelial cells. This screen was conducted as previously described (Fowler et al., Cell Host Microbe 23, 65-76 (2018)) using a pltB:gfp reporter. Transposon mutagenesis was conducted using a mariner transposon delivered by pSB4807, which was mobilized into the pltC:gfp S. typhi strain by conjugation using E. coli β-2163 Δnic35 as the donor strain. Mutants were selected by plating on LB agar plates containing 30 μg/ml chloramphenicol. A total of ˜150,000 mutants were collected and multiple aliquots of this library were stocked for subsequent use in the screen. For each iteration of the screen, five 15 cm dishes were seeded with 1×10⁷ Henle-407 cells each and grown for ˜24 h prior to infection using the transposon mutant library described above. Aliquots of the inoculum used for infection were collected for subsequent INSeq preparation (inoculum pool). 18 h post infection, the cells were washed three times with PBS, detached from plates using dilute trypsin and pelleted by centrifugation at 150×g for 5 min. Cells were lysed using a 5 min incubation in 0.1% sodium deoxycholate (in PBS) supplemented with 100 μg/ml DNase I to degrade and solubilize the genomic DNA released from lysed host cells. The lysate was centrifuged at 5000×g for 5 min to isolate S. typhi from the soluble cellular debris. The S. typhi-containing pellet was re-suspended in PBS and further purified from cellular debris using two spins at 150×g for 5 min (discarding the pellet fraction) and one spin at 5000×g for 5 min (discarding the supernatant). An aliquot of the recovered S. typhi was amplified by growth in LB at 37° C. and subsequently prepared for INSeq sequencing (post-infection pool) and the remainder was washed and diluted in PBS to a concentration of ˜2×10⁶ bacteria/ml for FACS. A total of ˜2×10⁷ S. typhi mutants were sorted according to their fluorescence intensity in the GFP channel (488 nm excitation, 515/20 with 505LP emission) using a BD FACS Aria II flow cytometer. The isolated low fluorescence and high fluorescence pools were amplified by growth in LB at 37° C. and subsequently prepared for INSeq sequencing. The screen was conducted using the same mutant library on two independent occasions, and the low fluorescence pools from these two sorts were pooled and re-sorted (re-sort of low fluorescence populations).

For INSeq sequencing, genomic DNA was extracted from each of the mutant pools, digested with MmeI (New England Biolabs) and barcoded samples were prepared for sequencing as described previously (van Opijnen et al., Nat. Methods 6, 767-772 (2009)). The purified 121 bp DNA products containing barcodes to identify the individual mutant pools were sequenced on an Illumina HiSeq2000 system. The sequencing data were analyzed using the INSeq_pipeline_v3 package (Goodman et al., Cell Host Microbe 6, 279-289 (2009)), which separated sequencing reads by pool, mapped/quantified insertions and grouped insertions by gene. For each pool, the total number of sequencing reads was normalized to be 2,176,000 (an average of 500/gene). To identify genes in which insertions were enriched in a statistically significant manner in one pool compared to another, a value of 50 (10% of the average number of reads per gene) was added to the normalized number of reads in both pools. Ratios of the log-transformed read numbers for the two pools were then calculated. Genes with values that were an average of more than two standard deviations from the mean over the two primary replicates of the screen and more than one standard deviation from the mean in all three sorts were considered to be significantly enriched.

Analytical Flow Cytometry

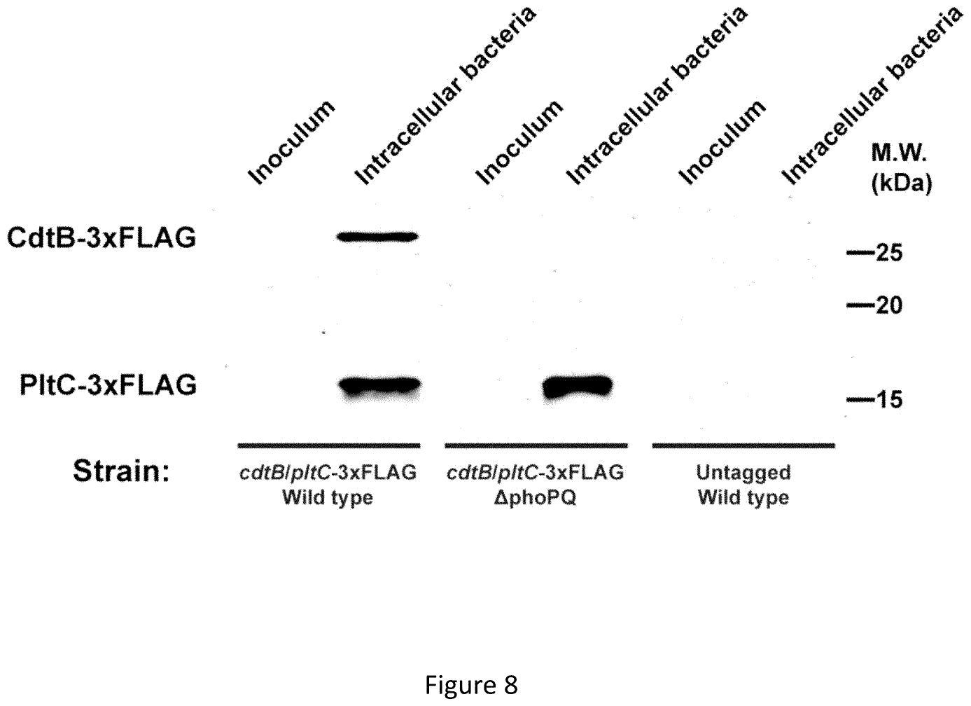

To probe pltB and pltC expression within infected cells at the single bacterium level, a previously developed flow cytometry-based assay was employed (Fowler et al., Cell Host Microbe 23, 65-76 (2018)). The indicated pltC:gfp and pltB:gfp strains carrying a plasmid driving constitutive mCherry expression were used to infect Henle-407 cells as described above. At 24 h post infection, bacteria were isolated and prepared for flow cytometry as described above for the FAST-INSeq screen. At this time point, which was chosen to capture a state of purine/pyrimidine starvation in the purM/pyrC mutants, very few instances of S. typhi within the host cell cytosol is found and thus virtually all S. typhi express high levels of both pltB and pltC in the wild-type strains (Fowler et al., Cell Host Microbe 23, 65-76 (2018)). For each sample the fluorescence intensity in the GFP channel (488 nm excitation, 515/20 with 505LP emission) was analyzed for at least 5000 mCherry-positive particles (532 nm excitation, 610/20 with 600LP emission) using a BD FACS Aria II flow cytometer. All samples were prepared and analyzed by flow cytometry in parallel. High and low fluorescence populations were defined based on the peaks observed in the wild-type samples for the given reporter strain. The intermediate population was defined as having a fluorescence intensity between and the low and high gates and the “greater than high fluorescence” population was defined as having a fluorescence intensity greater than the high fluorescence peak (>99.9% of particles in the wild-type sample). The gating strategy is provided in FIG. 8B.

The results of the experiments are now described.

### Many Salmonellae Encode AB₅ Toxins and “Orphan” B Subunits

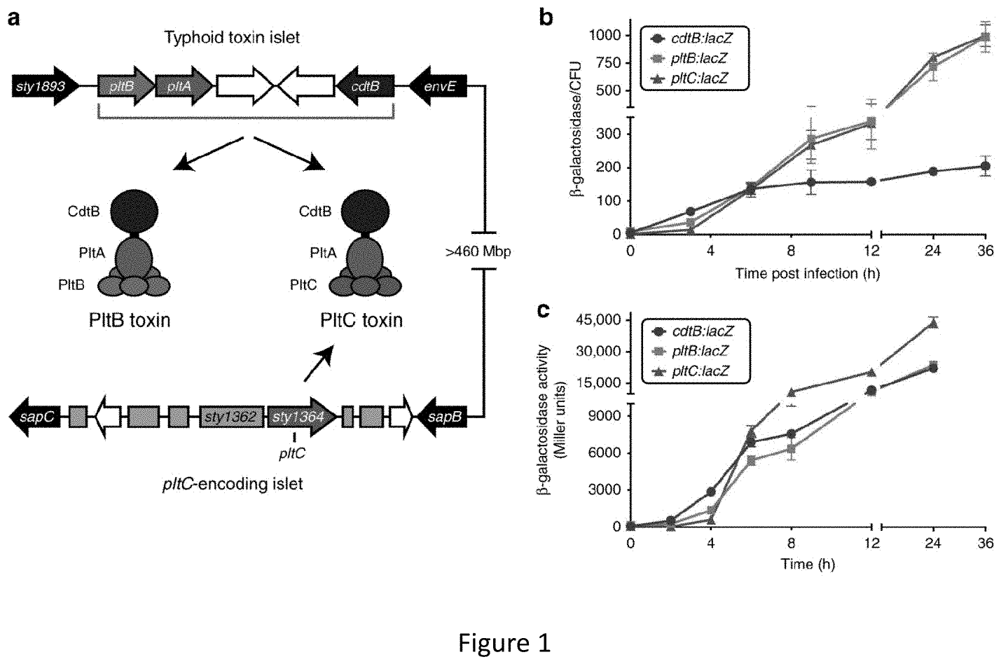

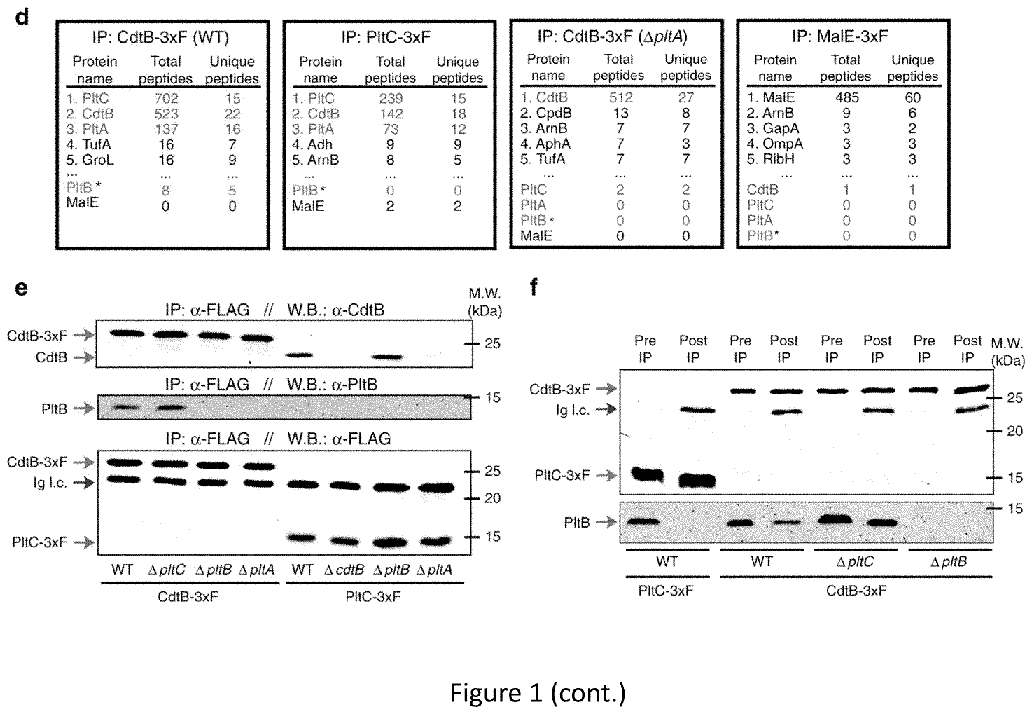

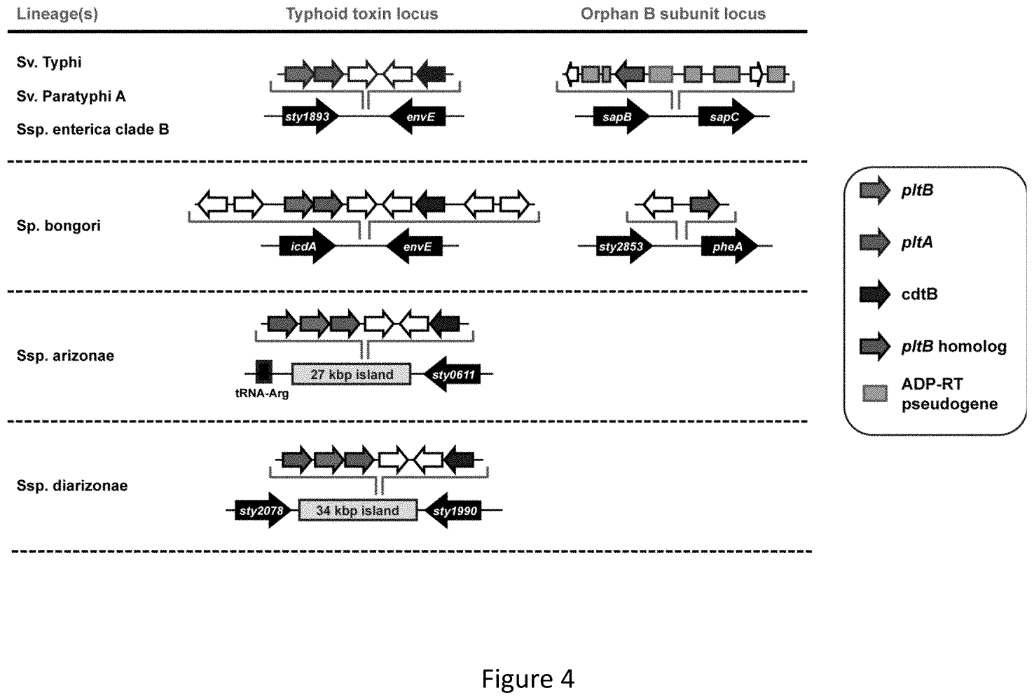

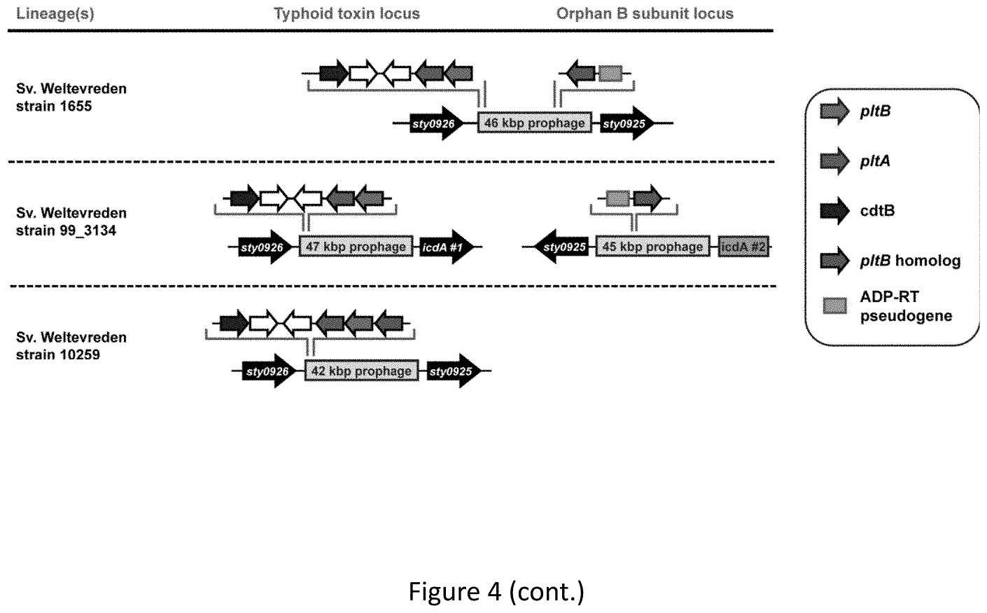

It was previously observed that, surprisingly, orthologous AB₅ toxin components encoded by different salmonellae are able to assemble into functional toxins (Gao et al., Nat. Microbiol. 2, 1592-1599 (2017)). Thus, it was examined whether there might be an evolutionarily-conserved phenomenon exploited by Salmonella to produce diversified toxins. To test this, the NCBI genome database was searched for salmonellae that encode both typhoid toxin (i.e. pltB, pltA and cdtB) as well as additional putative toxin-encoding genes homologous to typhoid toxin components. In agreement with previous reports (Miller et al., mBio 7, e02109-e02116 (2016); Rodriguez-Rivera et al., Gut Pathog. 7, 19 (2015)), it was found that the typhoid toxin islet is found in an assortment of Salmonella lineages and has a distribution that is consistent with having been transferred horizontally within the genus in multiple independent events. It was found that, in addition to the core toxin locus, almost every typhoid toxin-encoding strain that was identified encoded a second homolog of the pltB delivery subunit (FIG. 4). Remarkably, the genomic context of this putative second delivery subunit varies considerably among salmonellae. In some lineages, such as the arizonae and diarizonae subspecies, the additional pltB homolog is encoded immediately upstream of pltB, while in most cases, including the typhi and paratyphi serovars, it is found at a distant genome location as an “orphan” B subunit (FIG. 1A and FIG. 4). Importantly, although the majority of sequenced Salmonella strains do not encode typhoid toxin, no strains were identified that encode an orphan B subunit in the absence of typhoid toxin. The remarkable co-occurrence of these two genetic elements across different branches of the Salmonella genus supports the hypothesis that Salmonella may assemble alternative forms of typhoid toxins from orthologous components.

S. typhi Produces Two Distinct Forms of Typhoid Toxin

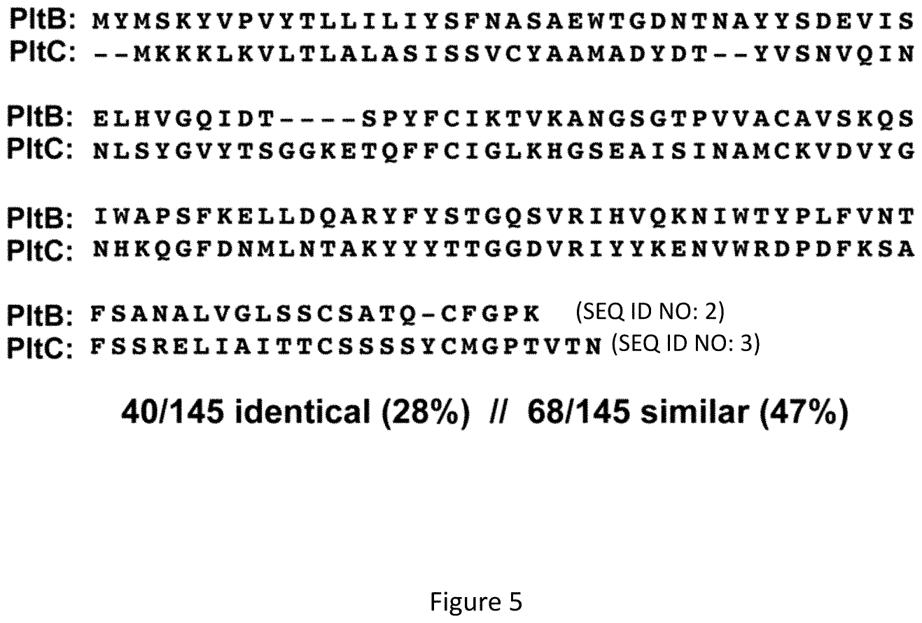

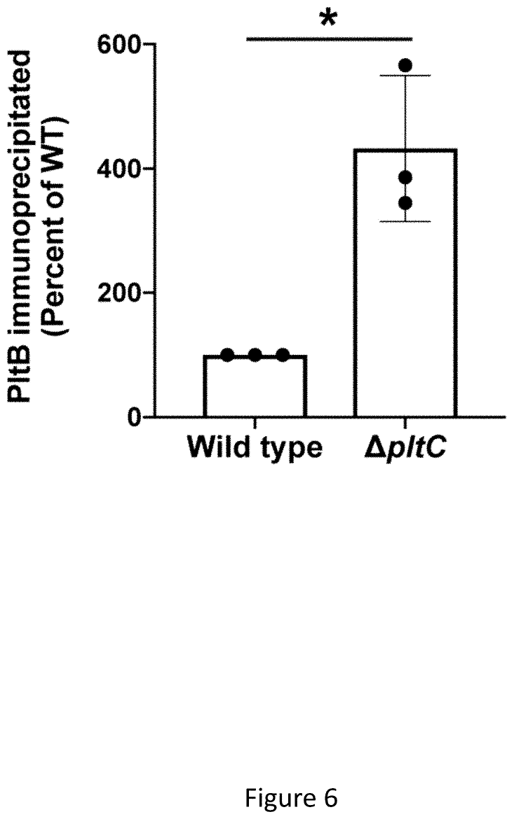

As alluded above, the S. typhi genome contains a gene that encodes a polypeptide that shares 28% amino acid sequence identity with PltB at a locus distant from the typhoid toxin islet (FIG. 1A, FIG. 4 and FIG. 5) (Rodriguez-Rivera et al., Gut Pathog. 7, 19 (2015)). This gene, sty1364, has been renamed pltC in accordance with the findings presented below. A thoroughly degraded ADP-ribosyltransferase pseudogene, sty1362, resides immediately upstream of pltC, suggesting that this locus encoded a complete AB₅-type toxin at some point in its evolutionary history (FIG. 4). It was examined whether PltC may associate with PltA and CdtB to produce an alternative form of typhoid toxin (FIG. 1A). In testing this hypothesis, it was found that pltC exhibited a similar pattern of expression to the genes encoding other components of typhoid toxin (Spano et al., Cell Host Microbe 3, 30-38 (2008); Haghjoo et al., Proc. Natl Acad. Sci. USA 101, 4614-4619 (2004); Fowler et al., Cell Host Microbe 23, 65-76 (2018)) in that, although undetectable under standard laboratory conditions, it was induced >100-fold following S. typhi infection of cultured human epithelial cells (FIG. 1B). Similarly, pltC expression was strongly induced in S. typhi grown in TTIM, a growth medium that mimics some aspects of the intracellular environment and is permissive for typhoid toxin expression in vitro (Fowler et al., Cell Host Microbe 23, 65-76 (2018)) (FIG. 1C). Using affinity chromatography coupled to LC-MS/MS and western blot analysis an interaction between PltC and both CdtB and PltA was readily detected after S. typhi growth in TTIM medium as well as after infection of cultured epithelial cells (FIG. 1D, FIG. 1E). Importantly, PltC did not interact with CdtB in the absence of PltA, indicating that similar to the holotoxin assembled with PltB, PltC forms a complex with CdtB only through its interaction with PltA (FIG. 1D and FIG. 1E). Although most AB₅ toxins employ a homopentameric delivery platform (Merritt et al., Curr. Opin. Struct. Biol. 5, 165-171 (1995)), pertussis toxin is comprised of a heteropentameric delivery platform assembled from different but structurally related B subunits (Stein et al., Structure 2, 45-57 (1994); Locht et al., FEBS J. 278, 4668-4682 (2011)). Because PltB is not efficiently detected using the LC-MS/MS protocol even in purified toxin preparations, it was unable to determine whether PltB and PltC form heteromeric delivery platforms using this approach. Therefore, to explore this issue immunoprecipitation coupled with western blot analysis was employed using an anti-PltB antibody in S. typhi-infected cells and in S. typhi grown in TTIM (FIG. 1E and FIG. 1F). Under both of these conditions, PltB was readily detected in samples affinity purified from a tag present in CdtB, but was undetectable in parallel samples purified from a tag present in PltC. These results indicate that, most likely, typhoid toxin does not exhibit a single heteromeric B subunit architecture but rather it is assembled in two alternative configurations with PltB or PltC as its homopentameric subunit (FIG. 1E and FIG. 1F). An increase in the amount of PltB that co-immunoprecipitated with CdtB in a ΔpltC strain compared to wild type was also observed, indicating that, in the absence of PltC, more PltB-containing toxin is assembled thus suggesting that the two delivery subunits compete for their association to the active subunits (FIG. 1F and FIG. 6). Following growth in TTIM, there also appears to be more PltB in the whole cell lysates in the ΔpltC mutant strain compared to wild type, which might indicate that free B subunits are degraded more readily than those incorporated into the toxin (FIG. 1F). Collectively, these data indicate that upon infection of human cells S. typhi assembles two distinct typhoid toxins with the same enzymatic “A” subunits but distinct delivery platforms or “B” subunits (FIG. 1A).

### The Two Typhoid Toxins Exhibit Significant Functional Differences

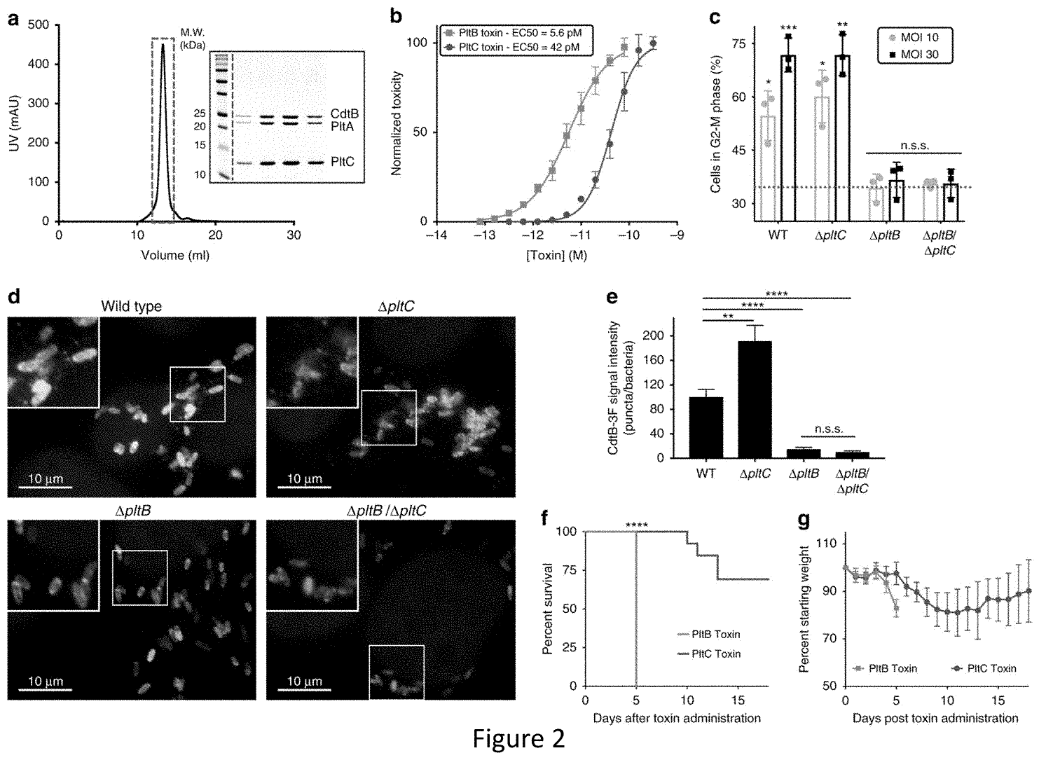

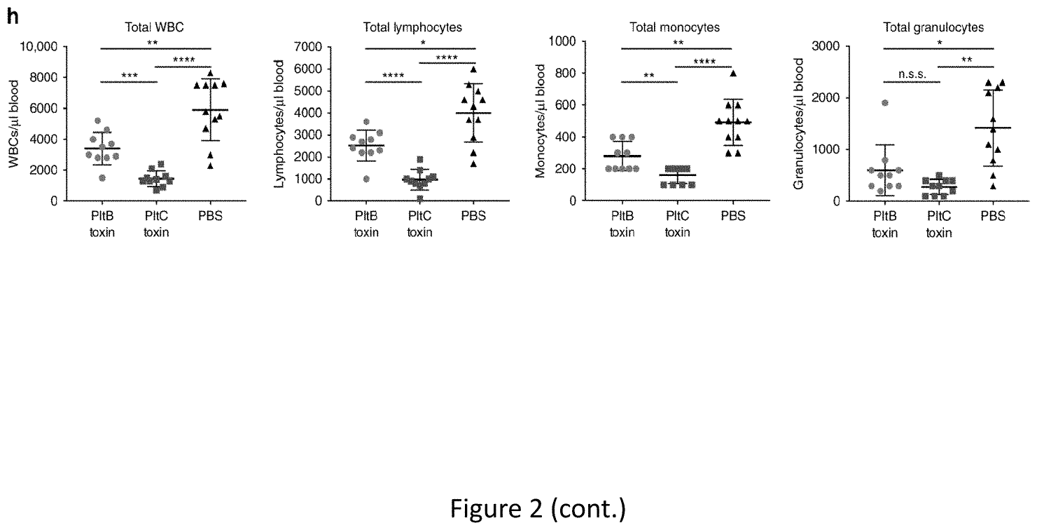

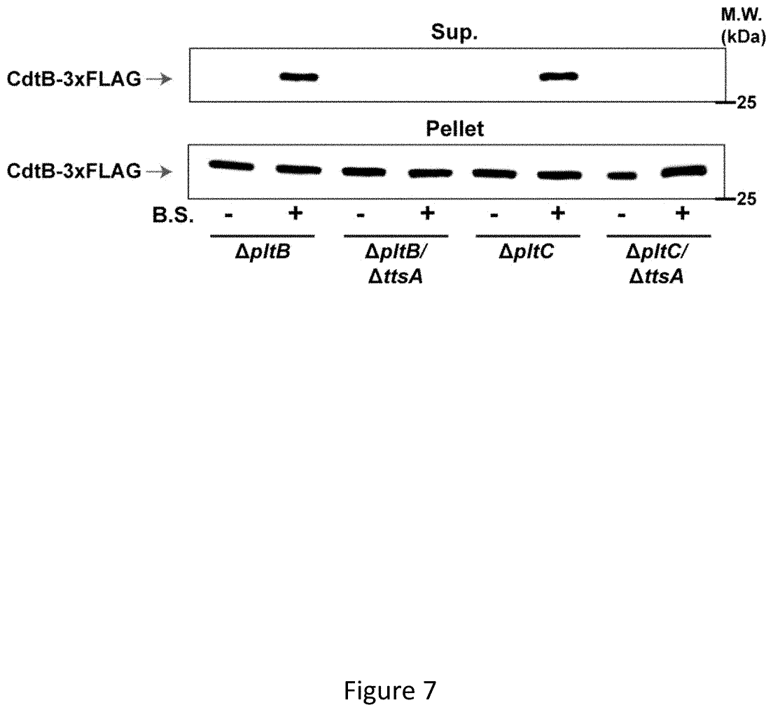

To assess the function of the typhoid toxin assembled with PltC the ability of the purified toxin to intoxicate cultured cells was examined as measured by cell cycle arrest at G2/M due to the DNA damage inflicted by the CdtB subunit (FIG. 2A and FIG. 2B) (Spano et al., Cell Host Microbe 3, 30-38 (2008); Haghjoo et al., Proc. Natl Acad. Sci. USA 101, 4614-4619 (2004); Lara-Tejero et al., Science 290, 354-357 (2000)). It was found that PltC-typhoid toxin was able to intoxicate cultured epithelial cells in a similar fashion to the PltB version of the toxin although with a higher (˜7 fold) EC50. Interestingly, however, cultured cells infected with a strain that lacks pltC (exclusively producing PltB-typhoid toxin) were intoxicated in a manner indistinguishable to cells infected with wild type S. typhi, although cells infected with a ΔpltB mutant strain (exclusively producing PltC-typhoid toxin) did not exhibit detectable signs of intoxication (FIG. 2C). The observations that PltC-typhoid toxin is produced to significant levels during infection (FIG. 1E) and is able to intoxicate when directly applied to cultured cells, but it does not intoxicate during bacterial infection suggested that the two alternative forms of the toxin might differ in their delivery mechanisms after their synthesis by intracellular S. typhi. It was previously shown that following its production, typhoid toxin is secreted from the bacterial periplasm into the lumen of the Salmonella-containing vacuole (SCV) by a specialized protein secretion mechanism involving a specialized muramidase that enables the toxin to cross to the trans side of the peptidoglycan (PG) layer, from where it can be released by various membrane-active agonists such as bile salts or anti-microbial peptides (Geiger et al., Nat. Microbiol. 3, 1243-1254 (2018)). It was found that both forms of typhoid toxin are released from the bacteria using this same mechanism (FIG. 7). It has been shown that after its secretion into the lumen of the SCV, typhoid toxin is packaged into vesicle transport intermediates that carry the toxin to the extracellular space, a process that is orchestrated by interactions of its B subunit PltB with specific luminal receptors (Spano et al., Cell Host Microbe 3, 30-38 (2008); Chang et al., Cell Host Microbe 20, 682-689 (2016)). Therefore, it was examined whether differences in receptor specificity between PltB and PltC may lead to differences in their intracellular transport pathways after bacterial infection. Cultured cells were infected with wild-type S. typhi or isogenic mutants carrying deletions in pltC, pltB, or both, and monitored the formation of CdtB-containing transport carriers using an immunofluorescence assay (FIG. 2D and FIG. 2E). It was found that toxin carriers were absent in cells infected with the ΔpltB strain although they were readily detected in cells infected with the ΔpltC mutant. These results indicate that the formation of the transport carriers is strictly dependent on PltB, presumably because the PltC version of typhoid toxin does not engage the sorting receptor. In fact, the level of transport carriers was measurably increased in cells infected with the ΔpltC S. typhi mutant, an indication that the absence of PltC leads to the assembly of higher levels of export-competent PltB-containing typhoid toxin. Collectively, these data indicate that the two typhoid toxins differ significantly with respect to their ability to engage the sorting receptors within the lumen of the SCV leading to substantial differences in the export of the toxins to the extracellular space, a pre-requisite for intoxication after bacterial infection of cultured cells. These results also suggest that, during infection, the two toxins may exert their function in different environments and may target different cells.

To gain insight into potential differences between the activities of the two forms of typhoid toxin, the consequences of systemically administering to C57BL/6 mice highly purified preparations of PltB- or PltC-typhoid toxins were evaluated. It was found that, although administering 2 μg of PltB-typhoid toxin was sufficient to kill all mice tested within five days, mice receiving 10 μg of PltC-Typhoid toxin (˜5-fold more) survived for at least 10 days and the majority survived the full course of the experiment (FIG. 2F). Furthermore, in contrast to PltB-typhoid toxin (Song et al., Nature 499, 350-354 (2013)), mice receiving the PltC-typhoid toxin showed no neurological symptoms, but did lose weight and showed signs of malaise and lethargy, although these symptoms were delayed and less severe than those observed in the PltB-typhoid toxin treated animals (FIG. 2G). Peak toxicity for PltC-typhoid toxin treated mice was observed between 8 and 13 days post-administration, after which the majority of treated animals fully recovered. Interestingly, despite eliciting fewer and milder overt symptoms compared the PltB-typhoid toxin treated mice, PltC-typhoid toxin caused a significantly greater reduction in the numbers of total white blood cells, lymphocytes and monocytes (FIG. 2H). Collectively, these data indicate that the two typhoid toxins preferentially target different cells/tissues. Therefore, producing two toxins variants confers functional versatility to typhoid toxin, presumably enabling S. typhi to expand the spectrum of host cell targets that it can engage.

### The Two Typhoid Toxins are Differentially Regulated

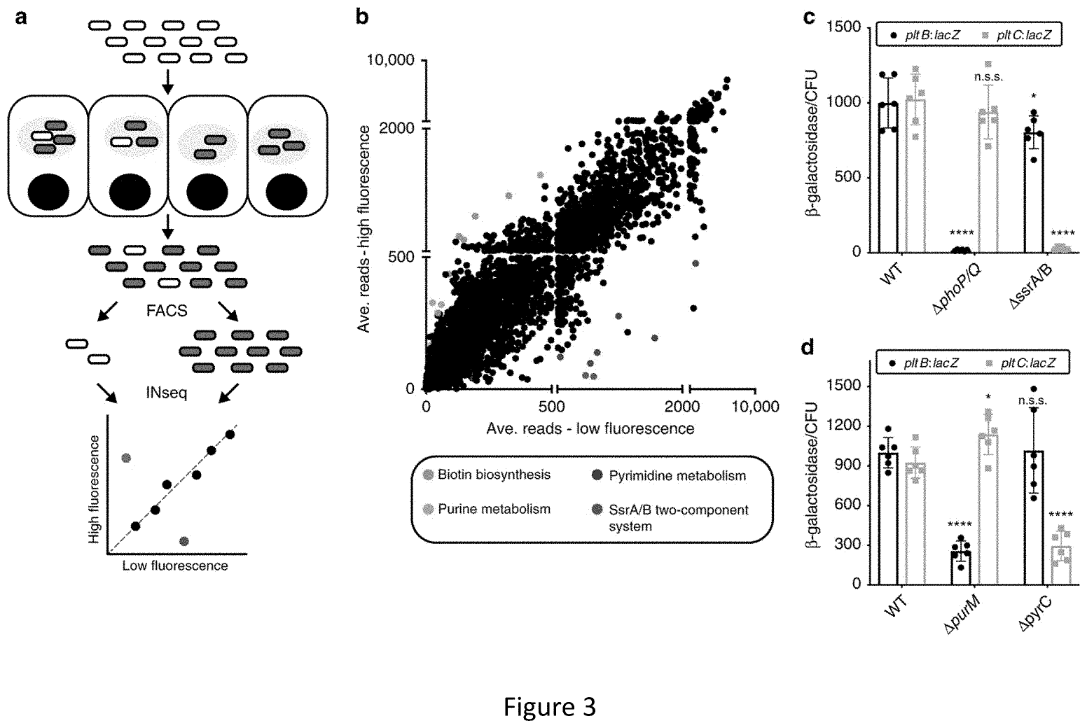

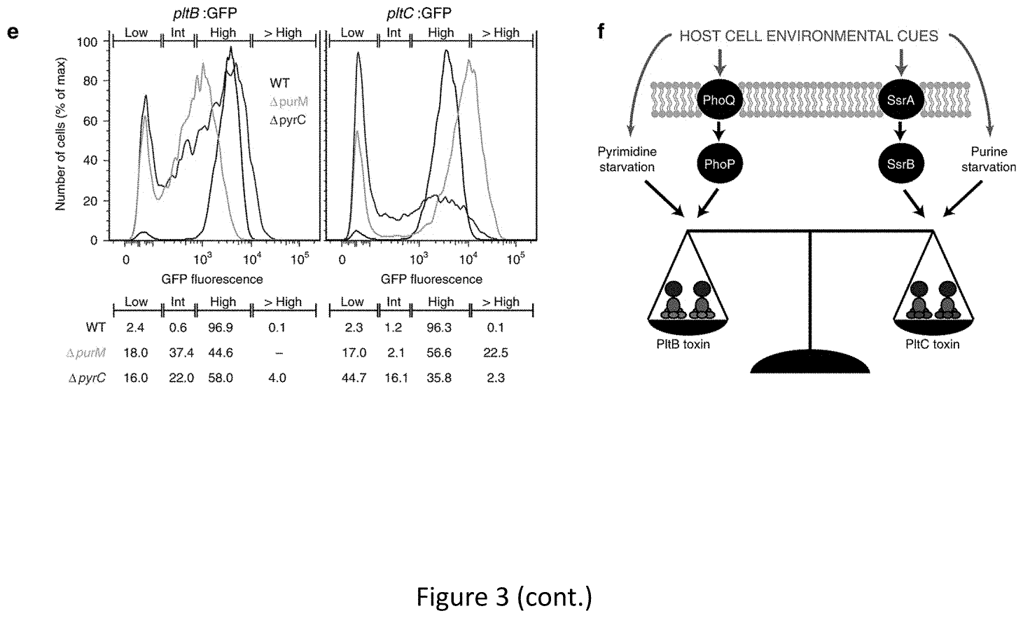

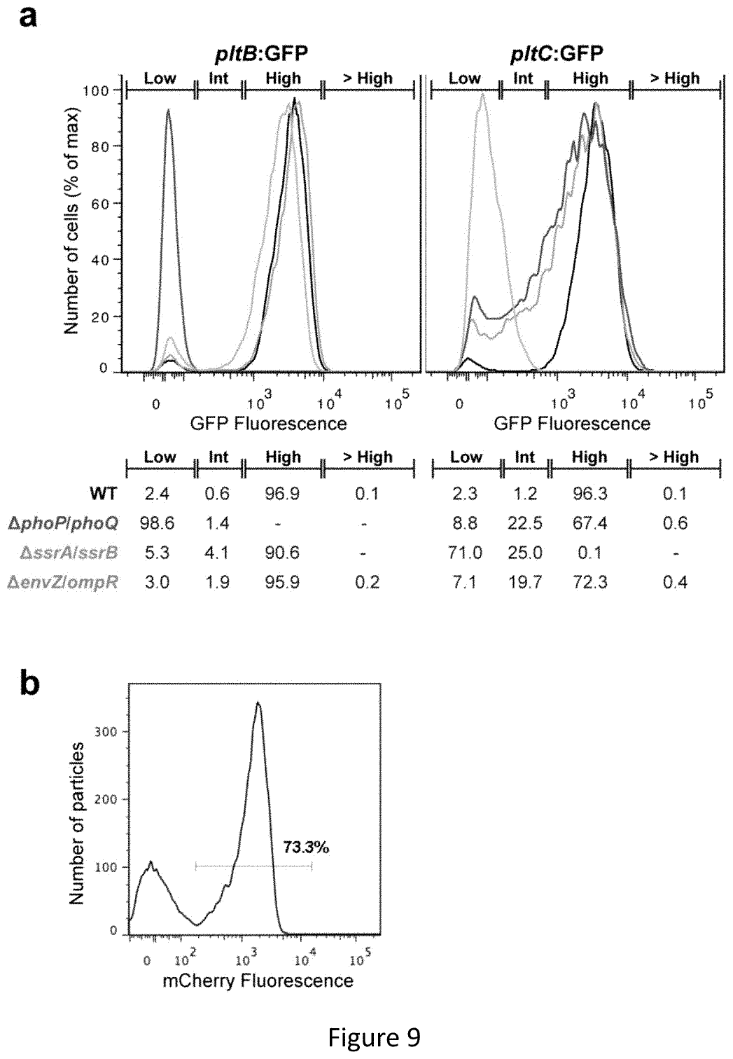

Given the substantially different functional properties exhibited by the two forms of typhoid toxin, it was reasoned that S. typhi might have evolved regulatory mechanisms to preferentially produce the different forms of the toxin under different conditions. Expression of the typhoid toxin locus genes (i.e. pltB, pltA and cdtB) is controlled by the PhoP/PhoQ (PhoPQ) two-component regulatory system (Fowler et al., Cell Host Microbe 23, 65-76 (2018)). However, it was found that in the absence of PhoPQ, pltC was robustly expressed during bacterial infection indicating that, despite exhibiting a similar intracellular expression pattern to the other typhoid toxin genes, the regulation of pltC expression must be distinct (FIG. 8 and FIG. 9). To decipher its regulatory network, FAST-INseq (Fowler et al., Cell Host Microbe 23, 65-76 (2018)) was used to screen for S. typhi genes that influence pltC expression within infected cultured cells (FIG. 3A). Cultured epithelial cells were infected with a library of S. typhi transposon mutants that encode a GFP reporter of pltC expression and, using fluorescence activated cell sorting (FACS), bacterial mutants that expressed pltC were separated from those that did not. Transposon insertion site sequencing (INseq)(Gawronski et al., Proc. Natl Acad. Sci. USA 106, 16422-16427 (2009); Goodman et al., Cell Host Microbe 6, 279-289 (2009); van Opijnen et al., Nat. Methods 6, 767-772 (2009)) was then used to identify transposon-disrupted genes that were over-represented in the bacterial population that did or did not express pltC, thus identifying candidate genes required for the regulation of pltC expression (FIG. 3B and Table 1). Notably, the most significantly enriched mutants that did not express pltC were insertions within ssrA and ssrB, which encode a two-component regulatory system (Table 1) (Ochman et al., Proc. Natl Acad. Sci. USA 93, 7800-7804 (1996); Shea et al., Proc. Natl Acad. Sci. USA 93, 2593-2597 (1996); Hensel et al., Mol. Microbiol. 30, 163-174 (1998)). This system is the master regulator of the expression of a type III protein secretion system encoded within Salmonella pathogenicity island 2, an essential Salmonella virulence factor that, like typhoid toxin, is selectively expressed by intracellular bacteria (Cirillo et al., Mol. Microbiol. 30, 175-188 (1998); Garmendia et al., Microbiology 149, 2385-2396 (2003); Fass et al., Curr. Opin. Microbiol. 12, 199-204 (2009)). S. typhi strains carrying deletion mutations in ssrA/ssrB, showed drastically reduced levels of pltC expression following infection of cultured epithelial cells, confirming the observations in the genetic screen (FIG. 3C and FIG. 9). Interestingly, phoP and phoQ were also identified amongst the mutants that yielded reduced pltC expression. Examination of pltC expression at the population and single bacterium levels (FIG. 3C, FIG. 8 and FIG. 9) revealed that, although most ΔphoPQ S. typhi express wild-type levels of pltC during infection, this mutant results in a larger population of intracellular S. typhi that fail to express pltC (2.3% wild type vs. 8.8% ΔphoPQ, see FIG. 9). Coupled with previous findings that PhoPQ can activate the expression of ssrA/ssrB, these data suggest that a small population of S. typhi require PhoPQ activation in order to stimulate ssrAB expression under these infection conditions (Fass et al., Curr. Opin. Microbiol. 12, 199-204 (2009); Bijlsma et al., Mol. Microbiol. 57, 85-96 (2005)). In stark contrast to what was observed for pltC, the absence of SsrA/SsrB had a negligible effect on pltB expression (FIG. 3C, FIG. 8 and FIG. 9). Collectively, these results suggest that the SsrA/SsrB two-component system is the principal activator of pltC expression during infection and indicate that the production of the two typhoid toxin delivery subunits is controlled by different, intracellularly-induced, global regulatory networks.

TABLE 1
Genes identified by FAST-INSeq as important
for pltC expression in infected human cells.

| | | | | | |
|---|---|---|---|---|---|
| | High | High | Low | Low | |
| | fluor: | fluor: | fluor: | fluor: | |
| Gene | insertions | reads | insertions | reads | Ratio^{a} |

Gene regulation

| | | | | | |
|---|---|---|---|---|---|
| ssrB | 22 | 48 | 27 | 936 | 20 |
| ssrA | 36 | 52 | 61 | 839 | 16 |
| phoP | 13 | 33 | 15 | 201 | 6 |
| phoQ | 26 | 62 | 27 | 335 | 5.5 |
| ompR | 14 | 73 | 13 | 244 | 3.5 |

Pyrimidine biosynthesis

| | | | | | |
|---|---|---|---|---|---|
| carA | 10 | 98 | 11 | 897 | 9 |
| carB | 35 | 194 | 33 | 1669 | 8.5 |
| pyrD | 21 | 139 | 20 | 979 | 7 |
| pyrE | 19 | 477 | 16 | 2705 | 5.5 |
| pyrB | 19 | 122 | 16 | 536 | 4.5 |
| pyrF | 10 | 40 | 7 | 188 | 4.5 |

Other

| | | | | | |
|---|---|---|---|---|---|
| proB | 20 | 307 | 20 | 2449 | 8 |
| proC | 12 | 216 | 11 | 1343 | 6 |
| rplS | 3 | 45 | 3 | 238 | 5.5 |
| proA | 27 | 798 | 24 | 3704 | 4.5 |
| ugpQ | 15 | 677 | 13 | 2652 | 4 |
| t3900 (bcsZ) | 25 | 260 | 19 | 864 | 3.5 |
| lysA | 33 | 1004 | 30 | 3497 | 3.5 |
| t4432 (ulaR) | 16 | 86 | 8 | 308 | 3.5 |
| vexA | 33 | 1099 | 25 | 3216 | 3 |
| t3052 | 13 | 549 | 10 | 1292 | 2.5 |
| tviB | 45 | 1990 | 35 | 5021 | 2.5 |

^{a}Ratio of normalized INSeq sequencing reads from transposon insertions within the indicated gene in the low fluorescence pool compared to the high fluorescence pool.

The screen also identified several mutants that led to increased pltC expression. Among these mutants were insertions within all of the genes required for the biosynthesis of the cofactor biotin (FIG. 3B and Table 2), which based on previous studies (Fowler et al., Cell Host Microbe 23, 65-76 (2018)), are likely to affect expression of pltC indirectly, by preventing the expansion of a population of cytosolic bacteria (i.e. located outside of the Salmonella containing vacuole) unable to express typhoid toxin. These results therefore suggest that, like the other typhoid toxin genes, pltC expression also requires the specific environment of the Salmonella containing vacuole. It was also found that insertions within genes required for purine biosynthesis resulted in increased pltC expression, while insertions within pyrimidine biosynthesis genes had the opposite effect (FIG. 3B, Table 1 and Table 2). This is particularly noteworthy since purine biosynthesis mutants result in reduced expression of pltB (Fowler et al., Cell Host Microbe 23, 65-76 (2018)). Follow up experiments exploring the expression of pltB and pltC in cultured cells infected with isogenic purine (ΔpurM) or pyrimidine (ΔpyrC) biosynthesis mutants confirmed the results of the screen and demonstrated that, in intracellular S. typhi, purine limitation favors pltC expression while pyrimidine limitation favors pltB expression (FIG. 3D and FIG. 3E). Further experiments will be required to decipher the nature of this regulation, however a wide range of regulatory mechanisms have been described involving nucleobases/nucleotides and second messengers derived from these molecules, many of which operate post transcriptional initiation (Valdivia et al., Nat. Chem. Biol. 13, 350-359 (2017); Dalebroux et al., Nat. Rev. Microbiol. 10, 203-212 (2012); Turnbough et al., Microbiol Mol. Biol. Rev. 72, 266-300 (2008); Bervoets et al., FEMS Microbiol. Rev. https://doi.org/10.1093/femsre/fuz001 (2019)). Given that levels of purines and pyrimidines are connected through their common use in DNA/RNA synthesis, it is tempting to speculate that the inverse effects observed for pltB and pltC expression may be due to a single regulatory molecule that exerts opposite effects on the expression of the two delivery subunits. Collectively, these results demonstrate that the relative expression levels of the two typhoid toxin delivery subunits are substantially altered in response to low nucleotide concentrations, and suggest that different nutrient availability may serve as a cue that enables S. typhi to adjust the balance of the two forms of typhoid toxin it produces within a given environment (FIG. 3F).

TABLE 2
Mutants identified by FAST-INSeq that led to an increased fraction
of S. Typhi expressing high levels of pltC in infected human cells.

| | | | | | |
|---|---|---|---|---|---|
| | High | High | Low | Low | |
| | fluor: | fluor: | fluor: | fluor: | |
| Gene | insertions | reads | insertions | reads | Ratio^{a} |

Biotin biosynthesis

| | | | | | |
|---|---|---|---|---|---|
| bioC | 12 | 289 | 7 | 46 | 6.5 |
| bioA | 31 | 679 | 20 | 131 | 5 |
| bioD | 16 | 436 | 9 | 90 | 5 |
| bioF | 27 | 929 | 19 | 195 | 5 |
| bioB | 33 | 639 | 22 | 150 | 4 |
| bioH | 19 | 1180 | 13 | 329 | 4 |

Purine metabolism

| | | | | | |
|---|---|---|---|---|---|
| purD | 13 | 328 | 4 | 25 | 13 |
| apt | 12 | 156 | 7 | 21 | 7.5 |
| purM | 17 | 321 | 7 | 60 | 5.5 |
| cpdA | 24 | 1432 | 16 | 455 | 3 |

Other

| | | | | | |
|---|---|---|---|---|---|
| t3263 | 9 | 198 | 4 | 15 | 13 |
| ssaQ | 23 | 142 | 6 | 16 | 9 |
| ilvY | 9 | 200 | 3 | 22 | 9 |
| tatB | 9 | 154 | 2 | 17 | 9 |
| rpmE2 | 12 | 167 | 7 | 19 | 9 |
| t3867 | 6 | 204 | 3 | 35 | 6 |
| t1909 | 18 | 279 | 9 | 46 | 6 |
| t2225 | 6 | 435 | 4 | 77 | 5.5 |
| t4475 | 6 | 244 | 4 | 46 | 5.5 |
| t0644 | 16 | 285 | 10 | 54 | 5.5 |
| sufl | 29 | 615 | 12 | 118 | 5 |
| hycF | 8 | 221 | 5 | 45 | 5 |
| t1519 | 25 | 263 | 14 | 61 | 4.5 |
| t2471 | 18 | 561 | 12 | 151 | 4 |
| t2824 | 7 | 403 | 6 | 112 | 4 |
| dacA | 18 | 411 | 9 | 106 | 4 |
| kdpB | 26 | 484 | 13 | 127 | 4 |
| t3076 | 12 | 448 | 8 | 137 | 3.5 |
| t4459 | 10 | 388 | 6 | 130 | 3 |

^{a}Ratio of normalized INSeq sequencing reads from transposon insertions within the indicated gene in the high fluorescence pool compared to the low fluorescence pool.

TABLE 3
List of bacterial strains used in this study.

| Strain | Relevant genotype | Reference |
|---|---|---|
| ISP2825 | Wild type S. Typhi | Galan et al., 1991, Infect Immun, |
| | | 59: 2901-2908 |
| SB1946 | cdtB-3xFLAG | Spano et al., 2008, Cell Host |
| | | Microbe, 3: 30-38 |
| SB2612 | pltB:lacZ | Fowler et al., 2018, Cell Host |
| | | Microbe, 23: 65-76 |
| SB2625 | pltB:lacZ ΔphoPQ | Fowler et al., 2018, Cell Host |
| | | Microbe, 23: 65-76 |
| SB2718 | pltB:gfp | Fowler et al., 2018, Cell Host |
| | | Microbe, 23: 65-76 |
| SB2900 | pltB:gfp ΔphoPQ | Fowler et al., 2018, Cell Host |
| | | Microbe, 23: 65-76 |
| SB2613 | cdtB:lacZ | Fowler et al., 2018, Cell Host |
| | | Microbe, 23: 65-76 |
| SB3211 | cdtB-3xFLAG, ΔpltC | This study |
| SB3222 | cdtB-3xFLAG, ΔpltA | This study |
| SB3226 | cdtB-3xFLAG, ΔpltB | This study |
| SB3385 | cdtB-3xFLAG, ΔpltB, | This study |
| | ΔpltC | |
| SB3207 | pltC-3xFLAG | This study |
| SB3406 | pltC-3xFLAG, ΔcdtB | This study |
| SB3221 | pltC-3xFLAG, ΔpltA | This study |
| SB3227 | pltC-3xFLAG, ΔpltB | This study |
| SB3230 | malE-3xFLAG (at pltC | This study |
| | locus) | |
| SB3219 | pltC-3xFLAG, cdtB- | This study |
| | 3xFLAG | |
| SB3229 | pltC-3xFLAG, cdtB- | This study |
| | 3xFLAG, ΔphoPQ | |
| SB3679 | cdtB-3xFLAG, ΔttsA, | This study |
| | ΔpltC | |
| SB3678 | cdtB-3xFLAG, ΔttsA, | This study |
| | ΔpltB | |
| SB3210 | ΔpltC | This study |
| SB3383 | ΔpltB | This study |
| SB3384 | ΔpltC, ΔpltB | This study |
| SB3407 | pltB:lacZ, ΔssrAB | This study |
| SB3654 | pltB:lacZ, ΔpurM | This study |
| SB3668 | pltB:lacZ, ΔpyrC | This study |
| SB3220 | pltC:lacZ | This study |
| SB3224 | pltC:lacZ, ΔphoPQ | This study |
| SB3409 | pltC:lacZ, ΔssrAB | This study |
| SB3655 | pltC:lacZ, ΔpurM | This study |
| SB3669 | pltC:lacZ, ΔpyrC | This study |
| SB3687 | pltB;gfp, ΔssrAB | This study |
| SB3675 | pltB:gfp, ΔenvZ/ompR | This study |
| SB3656 | pltB:gfp, ΔpurM | This study |
| SB3670 | pltB:gfp, ΔpyrC | This study |
| SB3400 | pltC:gfp | This study |
| SB3401 | pltC:gfp, ΔphoPQ | This study |
| SB3688 | pltC:gfp, ΔssrAB | This study |
| SB3676 | pltC:gfp, ΔenvZ/ompR | This study |
| SB3657 | pltC:gfp, ΔpurM | This study |
| SB3671 | pltC:gfp, ΔpyrC | This study |

Here, it is shown that S. typhi produces two different versions of typhoid toxin that share their enzymatic subunits but utilize alternative delivery subunits resulting in substantially different biological activities. In particular, these results indicate that PltB-typhoid toxin is more efficient at causing neurological symptoms, which are associated with increased lethality. In contrast, PltC-Typhoid toxin is more effective at targeting white blood cells as mice challenged with this form of typhoid toxin exhibited a more pronounced leukopenia. Therefore, by assembling toxins with different targeting mechanisms, Salmonella typhi may be able to target a broader array of cell types in different tissue environments.

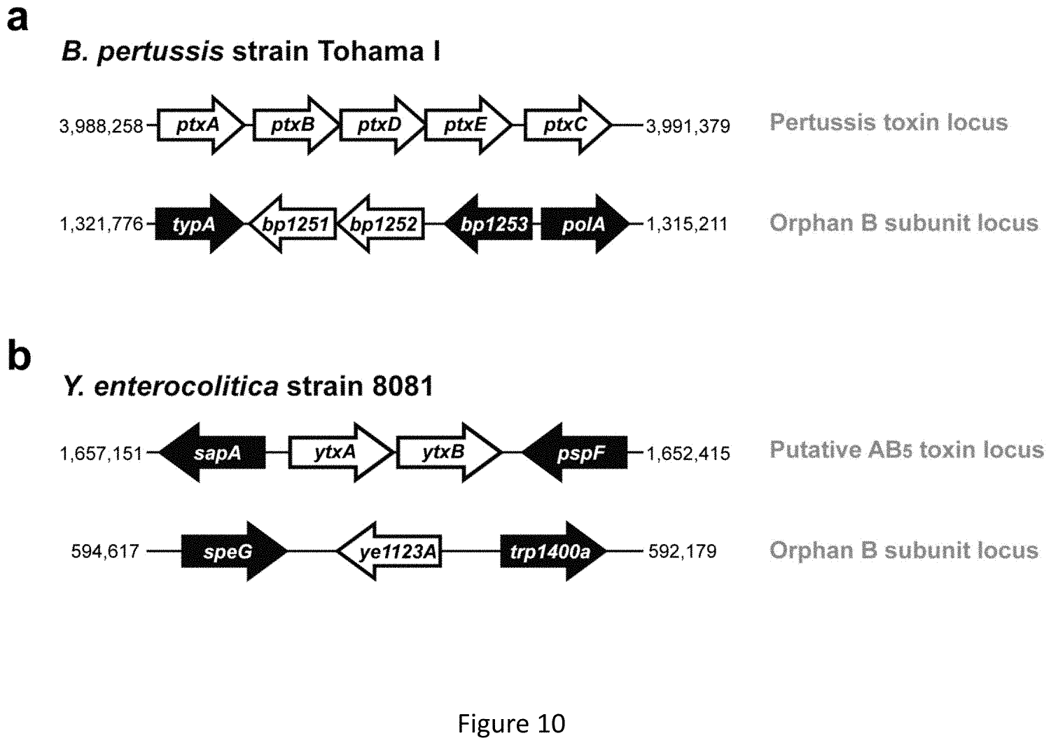

The typhoid toxin locus has a sporadic distribution in the Salmonella genus and is found in a range of different genome locations, often within prophage. Given these factors, it is noteworthy that virtually all typhoid toxin-encoding strains whose genomes have been sequenced also encode a second B subunit homolog (FIG. 4) (Miller et al., mBio 7, e02109-e02116 (2016); Rodriguez-Rivera et al., Gut Pathog. 7, 19 (2015); Miller et al., mBio 9, e00467-18 (2018)). This strongly suggests that producing multiple forms of typhoid toxin is not unique to the Typhi serovar, but rather is an integral aspect of typhoid toxin biology; indeed, genetic evidence suggests that an orthologous B subunit is also important for the function of the typhoid toxin produced by the Javiana serovar (Miller et al., mBio 9, e00467-18 (2018)). The distributions and genomic locations of these elements indicate that the acquisition of the core typhoid toxin islet and the second B subunit occurred in separate evolutionary events in several distinct Salmonella lineages, implying that producing functionally diverse typhoid toxins imparts a strong evolutionary advantage to ecologically diverse salmonellae. More broadly, other pathogens have also been identified that encode orphan B subunits that are homologous to components of an AB₅ toxin located elsewhere in their genomes, suggesting that the assembly of alternate toxins may be a more general feature of AB₅ toxins (FIG. 10). For example, in addition to the locus encoding pertussis toxin, Bordetella pertussis encodes two orphan orthologs of its B subunits at a distant genome location. Under certain conditions, these proteins have been reported to be co-synthesized and co-secreted with other pertussis toxin components (Luu et al., J. Proteom. 158, 43-51 (2017)). Here, it is shown that B subunits with significantly different amino acid sequences encoded by the same bacterium assemble with common A subunits into distinct toxins with different biological properties. This “lego-like” assembly of structurally-similar but functionally distinct components, which genomic evidence suggests is likely to be conserved in diverse bacterial lineages encoding different AB₅ toxins, suggests that the evolutionary expansion of the AB₅ class of toxins was likely fueled by the plasticity inherent to their structural design coupled with the functional versatility that can be achieved through combining homologous toxin components.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for inducing or enhancing an immune response comprising PltC or a PltC mutant.

2. The composition of claim 1, further comprising at least one polypeptide selected from the group consisting of:
a. PltA, or a PltA mutant;
b. PltB, or a PltB mutant; and
c. CdtB or a CdtB mutant.

3. The composition of claim 2, wherein the PltA mutant is PltA E133A, relative to SEQ ID NO: 1.

4. The composition of claim 2, wherein the PltB mutant is PltB S35A, relative to SEQ ID NO: 2.

5. The composition of claim 2, wherein the CdtB mutant is CdtB H160Q, relative to SEQ ID NO: 4; CdtB R119X, relative to SEQ ID NO: 4; CdtB H259X, relative to SEQ ID NO: 4; CdtB ΔCys269, relative to SEQ ID NO: 4; or CdtB C269X, relative to SEQ ID NO: 4.

6. The composition of claim 2, wherein the PltA mutant comprises any mutation in PltA that disrupts its enzymatic activity.

7. The composition of claim 2, wherein the CdtB mutant comprises any mutation in CdtB that disrupts its enzymatic activity.

8. The composition of claim 1, wherein the composition comprises PltA, or a PltA mutant; CdtB, or a CdtB mutant; and PltC or a PltC mutant.

9. The composition of claim 1, further comprising an antigen, wherein the composition enhances the immune response against the antigen.

10. The composition of claim 9, wherein the antigen is selected from the group consisting of a bacterial antigen, viral antigen, parasitic antigen, cancer antigen, tumor-associated antigen, and tumor-specific antigen.

11. The composition of claim 9, wherein the antigen is a S. typhi antigen or a S. paratyphi antigen.

12. The composition of claim 9, wherein the antigen is a bacterial polysaccharide antigen.

13. The composition of claim 9, wherein the antigen is Vi antigen.

14. The composition of claim 1, wherein the composition comprises a vaccine.

15. The composition of claim 1, wherein the composition comprises a bacterium.

16. A method of inducing an immune response in a subject, the method comprising administering the composition of claim 1 to the subject.

17. The method of claim 16, wherein the subject is currently infected with S. typhi or S. paratyphi and the composition induces an immune response against S. typhi or S. paratyphi.

18. The method of claim 16, wherein the subject is not currently infected with S. typhi or S. paratyphi and the composition induces an immune response against S. typhi or S. paratyphi.

19. A method of treating or preventing S. typhi infection in a subject, comprising administering the composition of claim 1 to the subject.

20. The method of claim 19, further comprising administering an antibiotic to the subject.

21. A method of treating or preventing a disease or disorder associated with an antigen in a subject, comprising administering the composition of claim 9 to the subject.

22. The method of claim 21, wherein the disease or disorder is at least one selected from the group consisting of cancer, a bacterial infection, a viral infection, and a parasitic infection.

23. An inhibitor composition useful for treating or preventing S. typhi or S. paratyphi infection, wherein the inhibitor composition inhibits PltC.

24. The inhibitor composition of claim 23, wherein the inhibitor composition comprises an antibody that specifically binds to PltC.

25. A method of treating a subject infected with S. typhi, the method comprising administering to the subject an inhibitor composition of claim 23.

26. The method of claim 25, further comprising administering an antibiotic to the subject.

27. A method of diagnosing an S. typhi or S. paratyphi infection in a subject in need thereof, the method comprising:
a. determining the level of PltC in a biological sample of the subject,
b. comparing the level of PltC with level in a comparator control, and
diagnosing the subject with an infection by S. typhi or S. paratyphi when the level of PltC is significantly different when compared with the level in the comparator control.

28. The method of claim 27, wherein the level of PltC in the biological sample is determined by measuring the level of PltC mRNA in the biological sample.

29. The method of claim 27, wherein the level of PltC in the biological sample is determined by measuring the level of PltC polypeptide in the biological sample.

30. The method of claim 27, wherein the comparator control is at least one selected from the group consisting of: a positive control, a negative control, a historical control, a historical norm, or the level of a reference molecule in the biological sample.

31. The method of claim 27, further comprising the step of administering a therapy to the subject to treat the infection.

32. A method of diagnosing an S. typhi or S. paratyphi infection in a subject in need thereof, the method comprising:
a. determining the level of antibody that specifically binds to PltC in a biological sample of the subject,
b. comparing the level of antibody that specifically binds to PltC with level in a comparator control, and
diagnosing the subject with an infection by S. typhi or S. paratyphi when the level of the antibody that specifically binds to PltC is significantly different when compared with the level in the comparator control.

33. The method of claim 32, wherein the comparator control is at least one selected from the group consisting of: a positive control, a negative control, a historical control, a historical norm, or the level of a reference molecule in the biological sample.

34. The method of claim 32, further comprising the step of administering a therapy to the subject to treat the infection.

35. A composition comprising a toxin-deficient S. typhi or S paratyphi bacterium, wherein the bacterium lacks PltC.

36. The composition of claim 35, wherein the composition is a vaccine and induces an adaptive immune response.

37. A method of immunizing a subject against S. typhi or S. paratyphi, the method comprising administering a composition of claim 35 to the subject.
